(19) United States
(12) Patent Application Publication
(10) Pub. No.: US 2025/0134810 A1
(43) Pub. Date: May 1, 2025

(54) LIPID PARTICLES, METHODS OF GENERATING SAME, AND METHODS OF USING SAME

(71) Applicants: Wisconsin Alumni Research Foundation, Madison, WI (US); WISYS TECHNOLOGY FOUNDATION, INC., Madison, WI (US)

(72) Inventors: Joseph Pierre, Waunakee, WI (US); Grace Lewis, Hastings, MN (US)

(73) Assignees: Wisconsin Alumni Research Foundation, Madison, WI (US); WISYS TECHNOLOGY FOUNDATION, INC., Madison, WI (US)

(21) Appl. No.: 18/932,825

(22) Filed: Oct. 31, 2024

Related U.S. Application Data
(60) Provisional application No. 63/594,799, filed on Oct. 31, 2023.

(51) Int. Cl. A61K 9/107; A61K 9/00; A61K 35/20

(52) U.S. Cl. CPC A61K 9/1075; A61K 9/0029; A61K 35/20

(57) ABSTRACT

Lipid particles, methods of generating same, and methods of using same. The lipid particles can be made from milk fat. The methods of generating the lipid particles can include melt fractionating milk fat, combining the melt-fractionated milk fat with a surfactant, obtaining the lipid particles by emulsifying the combined melt-fractionated milk fat and surfactant, and optionally, autoclaving the lipid particles. The methods of using the lipid particles can include parenterally administering the lipid particles to a subject in, for example, parenteral nutrition.

### CROSS-REFERENCE TO RELATED APPLICATIONS

Priority is hereby claimed to U.S. Provisional Application 63/594,799, filed Oct. 31, 2023, which is incorporated herein by reference in its entirety.

### FIELD OF THE INVENTION

The invention is directed to lipid particles, methods of generating same, and methods of using same. The invention in some embodiments is specifically directed to lipid particles comprising milk fat and methods of using same for parenteral nutrition.

### BACKGROUND

Parenteral nutrition (PN) is a clinical method of intravenous feeding that provides lifesaving nutrition support in patients who cannot feed via the gastrointestinal tract, due to trauma, surgery, intestinal inflammation or obstruction, or premature birth. PN is composed of dextrose, amino acids, vitamins, minerals, electrolytes, and lipids provided as emulsions. While lifesaving and used in over 300,000 patients a year in the U.S., the lipid emulsion formulations developed over the last 50 years remain problematic and are a major hurdle in optimizing metabolic requirements, growth, and preventing progressive hepatic complications. For instance, plant derived lipid fractions obtained from soybean, safflower, coconut, and olive oil have limited complexity, being dominated by either linoleic acid (soybean, safflower), oleic acid (olive oil), or medium chain triglycerides (coconut), resulting in efforts to mix plant oils together and better optimize formulations (Fell et al., 2015; Sadu Singh et al., 2020). Soybean oil is commonly used in many emulsion formulations despite containing phytosterols that likely induce hepatic toxicity. In European markets, extracted fish oils are included for their essential fatty acids (FAs), but have not been adopted in the U.S. The consensus in the field of surgical nutrition is that current lipids are sufficient but not optimal for patient care (Johnson et al., 2021). Furthermore, supply chain issues and the availability of raw lipid sources create frequent shortages.

Lipid formulations that overcome the foregoing problems are needed.

### SUMMARY OF THE INVENTION

One aspect of the invention is directed to methods of generating lipid particles.

In some versions the methods comprise generating a combined composition. In some versions, generating the combined composition comprises combining a milk fat composition comprising a solids portion comprising target milk fat with a surfactant.

In some versions, the methods further comprise generating a lipid-particle composition comprising lipid particles. In some versions, generating the lipid-particle composition comprises emulsifying the combined composition.

In some versions, the solids portion comprises the target milk fat in amount of at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, at least 96% w/w, at least 97% w/w, at least 98% w/w, or at least 99% w/w.

In some versions, the lipid composition comprises the solids portion in an amount of at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, at least 96% w/w, at least 97% w/w, at least 98% w/w, or at least 99% w/w.

In some versions, the lipid particles comprise triglycerides in an amount greater than 50% w/w, greater than 52.5% w/w, greater than 55% w/w, greater than 57.5% w/w, greater than 60%, greater than 62.5% w/w, greater than 65% w/w, greater than 67.5% w/w, greater than 70% w/w, greater than 72.5% w/w, greater than 75% w/w, greater than 77.5% w/w, greater than 80% w/w, greater than 82.5% w/w, or greater than 85% w/w of combined total of triglyceride, diglyceride, 1,2-diacylglyceryl-3-O-4′-(N,N,N-trimethyl)-homoserine, phosphatidylcholine, ether-linked phosphatidylcholine, fatty acyl ester of hydroxy fatty acid, free fatty acid, lysophosphatidylcholine, phosphatidylethanolamine, ether-linked phosphatidylethanolamine, lysophosphatidylethanolamine, non-hydroxy-fatty acid sphingosine ceramide, and sphingomyelin detected in positive ion mode of ultra-high-performance liquid chromatography/mass spectrometry/mass spectrometry.

In some versions, the lipid particles comprise a relative amount of any 2 or more, any 3 or more, any 4 or more, or each of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, and TG 12:0_14:0_16:0.

In some versions, the lipid particles comprise a relative amount of any 2 or more, any 3 or more, any 4 or more, any 5 or more, any 6 or more, any 7 or more, any 8 or more, any 9 or more, any 10 or more, any 11 or more, any 12 or more, any 13 or more, any 14 or more, any 15 or more, any 16 or more, any 17 or more, any 18 or more, any 19 or more, any 20 or more, any 21 or more, or each of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, TG 12:0_14:0_16:0, TG 16:0_18:0_18:1, TG 16:0_18:1_18:1, TG 16:0_16:0_18:1, TG 14:0_16:0_18:1, TG 12:0_16:0_18:1, TG 12:0_14:0_18:1, TG 14:0_18:1_18:1, TG 18:0_18:0_18:1, TG 16:0_17:0_18:1, TG 18:1_18:1_18:1, TG 12:0_12:1_18:1, TG 15:0_18:1_18:1, TG 15:0_16:0_18:1, TG 17:0_18:1_18:1, TG 17:0_18:0_18:1, TG 16:0_18:1_18:3, TG 16:0_18:0_18:0, TG 14:0_18:1_18:2, TG 14:0_16:0_18:0, TG 12:0_15:0_18:1, TG 15:0_16:0_18:0, and TG 16:0_17:0_18:0.

In some versions, the combined composition comprises the solids portion of the milk fat composition in an amount of at least 2.5% w/w, at least 5% w/w, at least 7.5% w/w, at least 10% w/w, at least 12.5% w/w, at least 15% w/w, at least 17.5% w/w, at least 20% w/w, at least 22.5% w/w, at least 25% w/w, at least 27.5% w/w, at least 30% w/w, at least 32.5% w/w, at least 35% w/w, at least 37.5% w/w, at least 40% w/w, at least 42.5% w/w, at least 45% w/w, at least 47.5% w/w, or at least 50% w/w.

In some versions, the generating the combined composition further comprises combining the milk fat composition with water.

In some versions, the combined composition comprises water in an amount of at least 50% w/w, at least 55% w/w, at least 60% w/w, at least 65% w/w, at least 70% w/w, at least 75% w/w, at least 80% w/w, at least 85% w/w, or at least 90% w/w.

In some versions, the combined composition comprises the surfactant in an amount of at least 0.01% w/w, at least 0.02% w/w, at least 0.03% w/w, at least 0.04% w/w, at least 0.05% w/w, at least 0.06% w/w, at least 0.07% w/w, at least 0.08% w/w, at least 0.09% w/w, at least 0.1% w/w, at least 0.2% w/w, at least 0.3% w/w, at least 0.4% w/w, at least 0.5% w/w and/or up to 0.5% w/w, up to 0.6% w/w, up to 0.7% w/w, up to 0.8% w/w, up to 0.9% w/w, up to 1% w/w, up to 2.5% w/w, up to 5% w/w, up to 7.5% w/w, up to 10% w/w, or more.

In some versions, greater than 90% by number of the lipid particles in the lipid-particle composition have a diameter of less than 500 nm.

In some versions, the emulsifying comprises high-pressure homogenization.

In some versions, the emulsifying generates an emulsified composition and the generating the lipid-particle composition further comprises size-filtering the emulsified composition with a filter comprising a pore size from 0.5 μm to 5.0 μm.

Some versions further comprise autoclaving the lipid particles.

Some versions further comprise generating the milk fat composition from a prior milk fat composition comprising the target milk fat and additional milk fat by removing the additional fat from the target milk fat.

In some versions, the generating the milk fat composition comprises melt fractionating the prior milk fat composition.

In some versions, the prior milk fat composition comprises at least one of anhydrous milk fat, butter oil, and ghee.

In some versions, the target milk fat comprises ruminant milk fat.

Another aspect of the invention is directed lipid particles. The lipid particles can be made according to the methods as described herein.

Another aspect of the invention is directed to methods of administering parenteral nutrition. In some versions, the methods comprise parenterally administering the lipid particles of the invention to a subject. In some versions, the subject has a condition comprising at least one of pancreatitis and hepatitis. In some versions, the administering results in a reduced pro-inflammatory response relative to administering an equivalent amount of lipid particles generated from vegetable fat.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic of an exemplary preparation of a milkfat-based intravenous fat emulsion. HMF: High melting fraction. LMF: low melting fraction.

FIG. 2. Schematic of the experimental steps carried out in the following examples.

FIG. 3. Low melting fraction (A) and high melting fraction (B) following fractionation of anhydrous milk fat (AMF) at 25° C.

FIG. 4. The relative abundance of various triglycerides for low melting fractions and high melting fractions at 15 and 25° C. (FIG. 4). 15C HMF, high melting fraction at 15° C. (HMF-15). 15C LMF, low melting fraction at 15° C. 25C HMF, high melting fraction at 25° C. 25C LMF, low melting fraction at 25° C. AMF Control, anhydrous milk fat. Shown are normalized log fold changes in concentration, wherein yellow is an increase in concentration and blue is a decrease.

FIG. 5. Relative abundance of triglyceride with three medium-chain, saturated (12:0) fatty acids (lauric acid) esterified to the glycerol backbone in AMF, a 15° C. high melting fraction (15H), a 15° C. low melting fraction (15 L), a 25° C. high melting fraction (25H), and a 25° C. low melting fraction (25 L).

FIG. 6. Fatty acid profile of a low melting fraction (LMF-25) of milk fat (AMF) vs. soy lipid (Intralipid® 20%). The milk fat had a much more diverse fatty acid profile. The bar to the left of the hash mark for each condition is LMF. The bar to the right of the hash mark for each condition is Intralipid® (IL).

FIG. 7. LC/MS/MS lipid analysis under positive mode and negative mode of a low melting fraction (LMF-25) of milk fat (AMF) and soy lipid (Intralipid® 20%). TG, triglyceride. DG, diglyceride. DGTS, 1,2-diacylglyceryl-3-O-4′-(N,N,N-trimethyl)-homoserine. PC, phosphatidylcholine. Ether PC, ether-linked phosphatidylcholine. FAHFA, fatty acyl ester of hydroxy fatty acid. FA, free fatty acid. LPC, lysophosphatidylcholine. PE, phosphatidylethanolamine. EtherPE, ether-linked phosphatidylethanolamine. LPE, lysophosphatidylethanolamine. Cer_NS, non-hydroxy-fatty acid sphingosine ceramide. SM, sphingomyelin.

FIG. 8. Annotated picture of a Nano DeBEE High-Pressure Homogenizer.

FIGS. 9A-9C. Size of milkfat IVFEs made according to a method as shown in FIG. 1 using 100 MPa (FIG. 9A), 200 MPa (FIG. 9B), and 300 MPa (FIG. 9C) in the high-pressure homogenization step.

FIG. 10. Sizes of milkfat IVFEs made according to a method as shown in FIG. 1 using 100 MPa in the high-pressure homogenization step. Graph indicates the size vs. intensity, where colors indicate the frequency of shown size vs. intensity plotted. Colors at the top of the legend are less frequent and grow more frequent at the lower shown symbols.

FIG. 11. Size stability of IVFEs over three months. Particle size over time for emulsions generated from 20% fractionated milkfat in water with 0.25, 0.5, or 1.0% polysorbate 80 emulsifier, and 150 or 300 MPa high-pressure homogenization for three passes. 0.25_150:0.25% polysorbate 80 processed at 150 MPa. 0.50_150:0.25% polysorbate 80 processed at 150 MPa. 1.0_150:0.25% polysorbate 80 processed at 150 MPa. 0.25_300:0.25% polysorbate 80 processed at 300 MPa. 0.50_300:0.25% polysorbate 80 processed at 300 MPa. 1.0_300:0.25% polysorbate 80 processed at 300 MPa.

FIG. 12. In vitro macrophage analysis. Milk lipid IVFEs (made according to FIG. 1) had a less inflammatory response (IL-1b, IL-6, and TNF-a) than soy lipid IVFEs (Intralipid® 20%) in stimulated macrophages (IL-1b, IL-6, and TNF-a), while inducing an anti-inflammatory response with IL-10.

FIGS. 13A-13C. Respiratory exchange ratio (RER) of mice administered milk lipid IVFEs (made according to FIG. 1) versus soy lipid IVFEs (Intralipid® 20%). FIG. 13A. RER time course.

FIG. 13B. RER daily average. FIG. 13C. RER average by phase. In FIGS. 13B and 13C, the bar to the left of the hash mark for each condition is milk lipid IVFE, and the bar to the right of the hash mark for each condition is soy lipid IVFE.

FIG. 14. Body, organ and tissue weights in mice administered milk lipid IVFEs (made according to FIG. 1) versus soy lipid IVFEs (Intralipid® 20%).

FIG. 15. Hematoxylin and eosin stain of pancreatic acinar tissue from mice administered milk lipid IVFEs (made according to FIG. 1) versus soy lipid IVFEs (Intralipid®).

FIG. 16. Hematoxylin and eosin stain of liver tissue from mice administered milk lipid IVFEs (made according to FIG. 1) versus soy lipid IVFEs (Intralipid® 20%).

### DETAILED DESCRIPTION OF THE INVENTION

One aspect of the invention is directed to methods of generating lipid particles. “Lipid particle(s)” as used herein refers to particle(s) comprising lipid. The particles of the invention can be in the form of micelles, such as oil-in-water micelles, and can be provided in a compositional form of an oil-in-water emulsion.

The methods of generating the lipid particles can comprise a step of generating a combined composition. The combined composition can generally comprise lipid in combination with a surfactant and water. The step of generating the combined composition can comprise combining a lipid composition with a surfactant and, optionally, water.

“Lipid composition” as used herein refers to any composition comprising, consisting of, or consisting essentially of lipid. “Lipid” and “fat” are used synonymously herein to refer to any substance comprising carbon, hydrogen, and, optionally, oxygen that is insoluble in water but soluble in non-polar solvents. The lipid can be in any form, such as triglycerides, diglycerides, monoglycerides, sterols, waxes, and free fatty acids, among others.

The lipid in the lipid composition preferably comprises milk fat, and more specifically target milk fat. “Milk fat” as used herein refers to fat contained in, isolated from, or derived from milk. “Target milk fat” as used herein refers to fat isolated or derived from milk. “Isolated from milk” refers to the isolation or separation of a given milk component (e.g., lipid or specific lipids) from at least a portion of at least one other component of milk (e.g., water, protein, sugar etc.). “Derived from milk” refers to a component that has been isolated from milk and chemically modified (e.g., esterified, etc.). Unless the context explicitly indicates otherwise, the general term “milk” as used herein refers to mammalian milk, as opposed to liquid extractions derived from plants, such as “almond milk” or “soy milk.” In some versions of the invention, the milk fat (e.g., target milk fat) in the lipid composition ruminant milk fat. The ruminant milk fat can be isolated or obtained from any ruminant, such as domesticated and wild bovines, goats, sheep, giraffes, deer, gazelles, and antelopes, among others. The target milk fat can be any type of fat or lipid or any combination of fats or lipids described herein.

The lipid composition and/or the combined composition can comprise a solids portion that comprises the lipid. “Solids portion” as used herein refers to the total combination of components of a given composition other than water. In addition to the lipid, the solids portion lipid composition and/or the combined composition can include protein such as casein and lactalbumin, carbohydrates such as lactose, minerals such as calcium and phosphorus, and other trace elements. If the lipid in the lipid composition and/or combined composition is a milk fat, the solids portion can include lipid solids (e.g., the milk fat) and non-lipid solids, such as typical non-lipid solids (“solids-not-fat” or “non-fat solids”) typically found in milk. Typical non-lipid solids found in milk include protein such as casein and lactalbumin, carbohydrates such as lactose, minerals such as calcium and phosphorus, and other trace elements. If the lipid in the lipid composition and/or combined composition is a target milk fat, the solids portion can have at least a portion of one or more non-lipid solids removed from the lipid solids present in the composition.

In various versions of the invention, the solids portion of the lipid composition (e.g., target milk fat composition) can comprise total lipid in amount of at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 91% w/w, at least 92% w/w, at least 93% w/w, at least 94% w/w, at least 95% w/w, at least 96% w/w, at least 97% w/w, at least 98% w/w, or at least 99% w/w. Amounts of 80% w/w or greater are preferred.

In various versions of the invention, the solids portion of the lipid composition (e.g., target milk fat composition) can comprise total lipid in amount of up to 5% w/w, up to 10% w/w, up to 15% w/w, up to 20% w/w, up to 25% w/w, up to 30% w/w, up to 35% w/w, up to 40% w/w, up to 45% w/w, up to 50% w/w, up to 55% w/w, up to 60% w/w, up to 65% w/w, up to 70% w/w, up to 75% w/w, up to 80% w/w, up to 85% w/w, up to 90% w/w, up to 91% w/w, up to 92% w/w, up to 93% w/w, up to 94% w/w, up to 95% w/w, up to 96% w/w, up to 97% w/w, up to 98% w/w, up to 99% w/w, or up to 99.9% or more.

In various versions of the invention, the solids portion of the lipid composition (e.g., target milk fat composition) can comprise target milk fat in amount of at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 91% w/w, at least 92% w/w, at least 93% w/w, at least 94% w/w, at least 95% w/w, at least 96% w/w, at least 97% w/w, at least 98% w/w, or at least 99% w/w. Amounts of 80% w/w or greater are preferred.

In various versions of the invention, the solids portion of the lipid composition (e.g., target milk fat composition) can comprise target milk fat in amount of up to 5% w/w, up to 10% w/w, up to 15% w/w, up to 20% w/w, up to 25% w/w, up to 30% w/w, up to 35% w/w, up to 40% w/w, up to 45% w/w, up to 50% w/w, up to 55% w/w, up to 60% w/w, up to 65% w/w, up to 70% w/w, up to 75% w/w, up to 80% w/w, up to 85% w/w, up to 90% w/w, up to 91% w/w, up to 92% w/w, up to 93% w/w, up to 94% w/w, up to 95% w/w, up to 96% w/w, up to 97% w/w, up to 98% w/w, up to 99% w/w, or up to 99.9% or more.

In various versions of the invention, the lipid composition (e.g., target milk fat composition) can comprise a solids portion in amount of at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, at least 96% w/w, at least 97% w/w, at least 98% w/w, or at least 99% w/w. Amounts 80% w/w or greater are preferred.

In various versions of the invention, the lipid composition (e.g., target milk fat composition) can comprise a solids portion in amount of up to 5% w/w, up to 10% w/w, up to 15% w/w, up to 20% w/w, up to 25% w/w, up to 30% w/w, up to 35% w/w, up to 40% w/w, up to 45% w/w, up to 50% w/w, up to 55% w/w, up to 60% w/w, up to 65% w/w, up to 70% w/w, up to 75% w/w, up to 80% w/w, up to 85% w/w, up to 90% w/w, up to 95% w/w, up to 96% w/w, up to 97% w/w, up to 98% w/w, up to 99% w/w, or up to 99.9% or more.

In various versions of the invention, the combined composition can comprise the solids portion of the lipid composition (e.g., milk fat composition) in an amount of at least 2.5% w/w, at least 5% w/w, at least 7.5% w/w, at least 10% w/w, at least 12.5% w/w, at least 15% w/w, at least 17.5% w/w, at least 20% w/w, at least 22.5% w/w, at least 25% w/w, at least 27.5% w/w, at least 30% w/w, at least 32.5% w/w, at least 35% w/w, at least 37.5% w/w, at least 40% w/w, at least 42.5% w/w, at least 45% w/w, at least 47.5% w/w, at least 50% w/w, at least 52.5% w/w, at least 55% w/w, at least 57.5% w/w, or at least 60% w/w. In various versions of the invention, the combined composition can comprise the solids portion of the lipid composition (e.g., milk fat composition) in an amount of up to 25% w/w, up to 27.5% w/w, up to 30% w/w, up to 32.5% w/w, up to 35% w/w, up to 37.5% w/w, up to 40% w/w, up to 42.5% w/w, up to 45% w/w, up to 47.5% w/w, up to 50% w/w, up to 52.5% w/w, up to 55% w/w, up to 57.5% w/w, up to 60% w/w, up to up to 62.5% w/w, up to 65% w/w, up to 67.5% w/w, up to 70% w/w, or more. Amounts from 5% w/w to 40% w/w are preferred.

In various versions of the invention, the combined composition can comprise water in an amount of at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, or at least 90% w/w. In various versions of the invention, the combined composition can comprise water in an amount up to up to 60% w/w, up to 65% w/w, up to 70% w/w, up to 75% w/w, up to 80% w/w, up to 85% w/w, up to 90% w/w, up to 95% w/w, up to 99% w/w, or more. Amounts from 60% w/w to 95% are preferred.

In some versions, the generating the combined composition further comprises combining the lipid composition (e.g., milk fat composition) with water. This step, and the amount of water added in this step, will depend on the amount of water in the lipid composition (e.g., milk fat composition) and the desired amount of water in the combined composition.

In some versions, the lipid particles comprise triglycerides in an amount greater than 50% w/w, greater than 52.5% w/w, greater than 55% w/w, greater than 57.5% w/w, greater than 60%, greater than 62.5% w/w, greater than 65% w/w, greater than 67.5% w/w, greater than 70% w/w, greater than 72.5% w/w, greater than 75% w/w, greater than 77.5% w/w, greater than 80% w/w, greater than 82.5% w/w, greater than 85% w/w of total lipids detected in positive ion mode of ultra-high-performance liquid chromatography/mass spectrometry/mass spectrometry. In some versions, the lipid particles comprise triglycerides in an amount up to 75% w/w, up to 77.5% w/w, up to 80% w/w, up to 82.5% w/w, up to 85% w/w, up to 87.5% w/w, up to 90% w/w, or more of total lipids detected in positive ion mode of ultra-high-performance liquid chromatography/mass spectrometry/mass spectrometry. In some versions, the lipid particles comprise triglycerides in an amount greater than 50% w/w, greater than 52.5% w/w, greater than 55% w/w, greater than 57.5% w/w, greater than 60%, greater than 62.5% w/w, greater than 65% w/w, greater than 67.5% w/w, greater than 70% w/w, greater than 72.5% w/w, greater than 75% w/w, greater than 77.5% w/w, greater than 80% w/w, greater than 82.5% w/w, or greater than 85% w/w of combined total of triglyceride, diglyceride, 1,2-diacylglyceryl-3-O-4′-(N,N,N-trimethyl)-homoserine, phosphatidylcholine, ether-linked phosphatidylcholine, fatty acyl ester of hydroxy fatty acid, free fatty acid, lysophosphatidylcholine, phosphatidylethanolamine, ether-linked phosphatidylethanolamine, lysophosphatidylethanolamine, non-hydroxy-fatty acid sphingosine ceramide, and sphingomyelin detected in positive ion mode of ultra-high-performance liquid chromatography/mass spectrometry/mass spectrometry. In some versions, the lipid particles comprise triglycerides in an amount up to 75% w/w, up to 77.5% w/w, up to 80% w/w, up to 82.5% w/w, up to 85% w/w, up to 87.5% w/w, up to 90% w/w, or more of combined total of triglyceride, diglyceride, 1,2-diacylglyceryl-3-O-4′-(N,N,N-trimethyl)-homoserine, phosphatidylcholine, ether-linked phosphatidylcholine, fatty acyl ester of hydroxy fatty acid, free fatty acid, lysophosphatidylcholine, phosphatidylethanolamine, ether-linked phosphatidylethanolamine, lysophosphatidylethanolamine, non-hydroxy-fatty acid sphingosine ceramide, and sphingomyelin detected in positive ion mode of ultra-high-performance liquid chromatography/mass spectrometry/mass spectrometry. The positive ion mode of ultra-high-performance liquid chromatography/mass spectrometry/mass spectrometry can be performed as described in the following examples.

In some versions, the lipid particles comprise a greater relative amount of any 1 or more, any 2 or more, any 3 or more, any 4 or more, any 5 or more, any 6 or more, any 7 or more, any 8 or more, any 9 or more, any 10 or more, any 11 or more, any 12 or more, any 13 or more, any 14 or more, any 15 or more, any 16 or more, any 17 or more, any 18 or more, any 19 or more, any 20 or more, any 21 or more, any 22 or more, any 23 or more, any 24 or more, any 25 or more, any 26 or more, or each of the following triglycerides compared to Intralipid® 20 lipid particles as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, TG 12:0_14:0_16:0, TG 16:0_18:0_18:1, TG 16:0_18:1_18:1, TG 16:0_16:0_18:1, TG 14:0_16:0_18:1, TG 12:0_16:0_18:1, TG 12:0_14:0_18:1, TG 14:0_18:1_18:1, TG 18:0_18:0_18:1, TG 16:0_17:0_18:1, TG 18:1_18:1_18:1, TG 12:0_12:1_18:1, TG 15:0_18:1_18:1, TG 15:0_16:0_18:1, TG 17:0_18:1_18:1, TG 17:0_18:0_18:1, TG 16:0_18:1_18:3, TG 16:0_18:0_18:0, TG 14:0_18:1_18:2, TG 14:0_16:0_18:0, TG 12:0_15:0_18:1, TG 15:0_16:0_18:0, and TG 16:0_17:0_18:0. The positive ion mode of ultra-high-performance liquid chromatography/mass spectrometry/mass spectrometry can be performed as described in the following examples.

In some versions, the lipid particles comprise a relative amount of any 2 or more, any 3 or more, any 4 or more, or each of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, and TG 12:0_14:0_16:0. The positive ion mode of ultra-high-performance liquid chromatography/mass spectrometry/mass spectrometry can be performed as described in the following examples.

In some versions, the lipid particles comprise a relative amount of any 2 or more, any 3 or more, any 4 or more, any 5 or more, any 6 or more, any 7 or more, any 8 or more, any 9 or more, any 10 or more, any 11 or more, any 12 or more, any 13 or more, any 14 or more, any 15 or more, any 16 or more, any 17 or more, any 18 or more, any 19 or more, any 20 or more, any 21 or more, or each of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, TG 12:0_14:0_16:0, TG 16:0_18:0_18:1, TG 16:0_18:1_18:1, TG 16:0_16:0_18:1, TG 14:0_16:0_18:1, TG 12:0_16:0_18:1, TG 12:0_14:0_18:1, TG 14:0_18:1_18:1, TG 18:0_18:0_18:1, TG 16:0_17:0_18:1, TG 18:1_18:1_18:1, TG 12:0_12:1_18:1, TG 15:0_18:1_18:1, TG 15:0_16:0_18:1, TG 17:0_18:1_18:1, TG 17:0_18:0_18:1, TG 16:0_18:1_18:3, TG 16:0_18:0_18:0, TG 14:0_18:1_18:2, TG 14:0_16:0_18:0, TG 12:0_15:0_18:1, TG 15:0_16:0_18:0, and TG 16:0_17:0_18:0. The positive ion mode of ultra-high-performance liquid chromatography/mass spectrometry/mass spectrometry can be performed as described in the following examples.

In some versions, the lipid particles comprise a lower relative amount of any 1 or more, any 2 or more, any 3 or more, any 4 or more, any 5 or more, any 6 or more, any 7 or more, any 8 or more, any 9 or more, any 10 or more, or each of the following triglycerides compared to Intralipid® 20 lipid particles as detected in positive ion mode of ultra-high-performance liquid chromatography/mass spectrometry/mass spectrometry: TG 18:2_18:2_22:0, TG 18:1_18:2_22:0, TG 18:1_18:1_22:0, TG 18:1_18:2_18:2, TG 18:2_18:2_18:2, TG 18:1_18:2_18:3, TG 18:1_18:1_18:2, TG 18:0_18:1_18:1, TG 16:0_18:1_18:2, TG 16:0_18:2_18:3, and TG 18:2_18:3_18:3. The positive ion mode of ultra-high-performance liquid chromatography/mass spectrometry/mass spectrometry can be performed as described in the following examples.

In some versions, the lipid particles comprise any 1 or more, any 2 or more, any 3 or more, any 4 or more, any 5 or more, any 6 or more, any 7 or more, any 8 or more, any 9 or more, any 10 or more, any 11 or more, any 12 or more, any 13 or more, any 14 or more, any 15 or more, any 16 or more, any 17 or more, any 18 or more, any 19 or more, any 20 or more, any 21 or more, any 22 or more, any 23 or more, any 24 or more, any 25 or more, any 26 or more, or each of the lipids shown in Tables 6A-6F. The positive ion mode of ultra-high-performance liquid chromatography/mass spectrometry/mass spectrometry can be performed as described in the following examples.

In some versions, the lipid particles comprise any 2 or more, any 3 or more, any 4 or more, any 5 or more, any 6 or more, any 7 or more, any 8 or more, any 9 or more, any 10 or more, any 11 or more, any 12 or more, any 13 or more, any 14 or more, any 15 or more, any 16 or more, any 17 or more, any 18 or more, any 19 or more, any 20 or more, any 21 or more, any 22 or more, any 23 or more, any 24 or more, any 25 or more, any 26 or more, or each of the lipids shown in Tables 6A-6F in relative amounts with respect to each other within +/−10-fold, +/−5-fold, +/−2-fold, +/−95%, +/−90%, +/−85%, +/−80%, +/−75%, +/−70%, +/−75%, +/−70%, +/−65%, +/−60%, +/−55%, +/−50%, +/−45%, +/−40%, +/−35%, +/−30%, +/−25%, +/−20%, +/−15%, +/−10%, +/−5%, or +/−1% of the relative amounts between such lipids as shown in Tables 6A-6F, as detected in positive ion mode of ultra-high-performance liquid chromatography/mass spectrometry/mass spectrometry. The positive ion mode of ultra-high-performance liquid chromatography/mass spectrometry/mass spectrometry can be performed as described in the following examples.

The surfactant combined with the lipid composition (e.g., milk fat composition) in generating the combined composition can be any surfactant that can serve as an emulsifier in generating an emulsion from the combined composition.

The surfactant can be an amphiphilic compound that comprises a hydrophilic head and a hydrophobic tail. The hydrophilic head can comprise a polar, nonionic head group or an ionic head group. The ionic head group can be an anionic head group, a cationic head group, or a zwitterionic head group.

The nonionic head groups can include hydroxyl groups or other polar groups. Examples of surfactants that comprise a nonionic head group include long chain alcohols, such as cetyl alcohol, stearyl alcohol, cetostearyl alcohol (consisting predominantly of cetyl and stearyl alcohols), and oleyl alcohol; polyoxyethylene glycol alkyl ethers (Brij), such as those having the formula CH₃-(CH₂)₁₀₋₁₆—(O—C₂H₄)₁₋₂₅—OH, including octaethylene glycol monododecyl ether and pentaethylene glycol monododecyl ether, among others; polyoxypropylene glycol alkyl ethers, such as those having the formula CH₃—(CH₂)₁₀₋₁₆—(O—C₃H₆)₁₋₂₅—O; glucoside alkyl ethers, such as those having the formula CH₃—(CH₂)₁₀₋₁₆—(O-Glucoside)₁₋₃-OH, including decyl glucoside, lauryl glucoside, and octyl glucoside, among others; polyoxyethylene glycol octylphenol ethers, such as those having the formula C₈H₁₇—(C₆H₄)—(O—C₂H₄)₁₋₂₅—OH, including Triton X-100, among others; polyoxyethylene glycol alkylphenol ethers, such as those having the formula C₉H₁₉—(C₆H₄)—(O—C₂H₄)₁₋₂₅—OH, including nonoxynol-9, among others; glycerol alkyl esters, such as glyceryl laurate, among others; polyoxyethylene glycol sorbitan alkyl esters, such as polysorbate (e.g., polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate)), among others; sorbitan alkyl esters, such as Spans, among others; cocamide MEA; cocamide DEA; codecyldimethylamine oxide; block copolymers of polyethylene glycol and polypropylene glycol, such as poloxamers, among others; and polyethoxylated tallow amine (POEA).

The anionic head groups can include sulfate, sulfonate, phosphate, and/or carboxylate groups, among others. Examples of surfactants that comprise an anionic head group include alkyl sulfates, such as ammonium lauryl sulfate, sodium lauryl sulfate (SDS, sodium dodecyl sulfate), alkyl-ether sulfates such as sodium laureth sulfate, and sodium myreth sulfate, among others. Examples of surfactants that comprise an anionic head group also include sulfonates, such as dioctyl sodium sulfosuccinate, perfluorooctanesulfonate (PFOS), perfluorobutanesulfonate, linear alkylbenzene sulfonates (LABs), and carboxylates, among others. Carboxylates include alkyl carboxylates, such as fatty acids and salts thereof. Examples of carboxylates include sodium stearate, sodium lauroyl sarcosinate, and carboxylate-based fluorosurfactants, such as perfluorononanoate, and perfluorooctanoate (PFOA or PFO). Other examples of anionic surfactants include cocoyl isethionate, sodium dodecylbenzinesulfonate, and sodium isethionate.

The cationic head groups can include pH-dependent primary, secondary, or tertiary amines and permanently charged quaternary ammonium cations, among others. Primary amines become positively charged at pH<10, secondary amines become positively charged at pH<4. An example of a pH-dependent amine is octenidine dihydrochloride. Permanently charged quaternary ammonium cations include alkyltrimethylammonium salts, such as cetyl trimethylammonium bromide (CTAB, hexadecyl trimethyl ammonium bromide), cetyl trimethylammonium chloride (CTAC), cetylpyridinium chloride (CPC), benzalkonium chloride (BAC), benzethonium chloride (BZT), 5-Bromo-5-nitro-1,3-dioxane, dimethyldioctadecylammonium chloride, cetrimonium bromide, and dioctadecyldimethylammonium bromide (DODAB), among others.

Zwitterionic (amphoteric) surfactants have both cationic and anionic centers attached to the same molecule. The cationic center can be based on primary, secondary, or tertiary amines, quaternary ammonium cations, or others. The anionic part can include sulfonates, as in CHAPS (3-[(3-Cholamidopropyl)dimethylammonio]-1-propanesulfonate), or sultaines, as in cocamidopropyl hydroxysultaine. Other examples of zwitterionic head groups include betaines, such as cocamidopropyl betaine, and choline-phosphates, such as those occurring in lecithin, among others.

For ionic head groups, the counter-ion can be monoatomic/inorganic or polyatomic/organic. Monoatomic/inorganic cationic counter-ions include metals, such as the alkali metals, alkaline earth metals, and transition metals. Monoatomic/inorganic anionic counter-ions include the halides, such as chloride (Cl⁻), bromide (Br⁻), and iodide (I⁻). Polyatomic/organic cationic counter-ions include ammonium, pyridinium, and triethanolamine (TEA), among others. Polyatomic/organic anionic counter-ions include tosyls, trifluoromethanesulfonates, and methylsulfate, among others.

The hydrophobic tail of the surfactant can include a linear, branched, or aromatic hydrocarbon chain. The hydrocarbon chain can have any number of carbon atoms suitable to render it hydrophobic. The number of carbon atoms can include from 9 to 30 carbon atoms, from 10 to 20 carbon atoms, or from 12 to 18 carbon atoms. Such carbon atoms can be saturated, unsaturated, straight-chained, branched, or cyclic.

The surfactant can comprise a natural surfactant and/or a synthetic surfactant. As used herein, “natural surfactant” refers to a saponified animal or vegetable fat or purified components thereof. “Synthetic surfactant” refers to a surfactant that is not a natural surfactant. The animal or vegetable fat used to generate the natural surfactant can be a solid fat or a liquid fat (i.e., an oil). Examples of solid fats include lard, tallow, and vegetable shortening, among others. Examples of liquid fats include oils such as coconut oil, peanut oil, almond oil, palm oil, olive oil, and soybean oil, among others. Other suitable fats include apricot kernel, sweet almond, jojoba, evening primrose, wheat germ, avocado, shea butter, and coconut butter, among others. To generate the natural surfactant, the fats are saponified, i.e., hydrolyzed, with a strong base. Lye is a suitable strong base. Caustic soda (sodium hydroxide) and caustic potash (potassium hydroxide) are both examples of lye. Saponification of fat results in a saponified fat composition. The saponified fat composition can comprise fatty acids or salts thereof, glycerol, any cations remaining from the saponification, such as sodium and/or potassium, and/or any non-hydrolyzed fat. The sodium and potassium can be complexed with the fatty acid to form a fatty salt or can be free ions. The glycerol may or may not be removed from the saponified fat.

In various versions of the invention, the surfactant can be included in the combined composition in an amount of at least 0.01% w/w, at least 0.02% w/w, at least 0.03% w/w, at least 0.04% w/w, at least 0.05% w/w, at least 0.06% w/w, at least 0.07% w/w, at least 0.08% w/w, at least 0.09% w/w, at least 0.1% w/w, at least 0.2% w/w, at least 0.3% w/w, at least 0.4% w/w, at least 0.5% w/w and/or up to 0.5% w/w, up to 0.6% w/w, up to 0.7% w/w, up to 0.8% w/w, up to 0.9% w/w, up to 1% w/w, up to 2.5% w/w, up to 5% w/w, up to 7.5% w/w, up to 10% w/w, or more. In various versions of the invention, the surfactant can be included in the combined composition in an amount up to 0.6% w/w, up to 0.7% w/w, up to 0.8% w/w, up to 0.9% w/w, up to 1% w/w, up to 2.5% w/w, up to 5% w/w, up to 7.5% w/w, up to 10% w/w, up to 12.5% w/w, up to 15% w/w, up to 17.5% w/w, up to 20% w/w or more. Amounts from 0.05% to 2% are preferred.

The methods of generating the lipid particles of the invention can further comprise a step of emulsifying the combined composition to generate an emulsified composition. “Emulsifying” as used herein refers to any method suitable for emulsifying the lipid of the combined composition in the water of the combined composition, e.g., in the form of micelles in an oil-in-water emulsion. Any emulsifying method can be used. Examples include mixing, stirring, homogenization, microfluidization, sonication, etc.

Preferred emulsification methods include methods capable of generating lipid particles (e.g., micelles) having a diameter less than 1,000 nm, and more preferably less than, 900, 800, 700, 600, or 500 nm. Exemplary emulsification methods capable generating lipid particles of this size include high-pressure homogenization. In the form of micelles, the lipid particles of the invention are typically spherical in shape. Thus, the term “diameter” represents a true diameter of the lipid particles. In cases in which the lipid particles are not spherical in shape, the term “diameter” is defined according to maximum dimension.

In various versions of the invention, the emulsification is effective to generate a high proportion of lipid particles having a diameter less than 1,000 nm. For example, the lipid particles in various versions of the invention can have a diameter less than 1,000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, or less than 400 nm. The lipid particles in various versions of the invention can have a diameter greater than 1 nm, greater than 5 nm, greater than 25 nm, greater than 50 nm, greater than 75 nm, greater than 100 nm, greater than 150 nm, or greater than 200 nm. In various embodiments, the lipid particles having any of these stated values (e.g., less than 500 nm) or combination of stated values (e.g., less than 500 nm and greater than 200 nm) can constitute at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the total number lipid particles in a given composition, such as the emulsified composition or compositions obtained or derived therefrom. In various embodiments, the lipid particles having any of these stated values (e.g., less than 500 nm) or combination of stated values (e.g., less than 500 nm and greater than 200 nm) can constitute up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, or up to 100% of the total number lipid particles in a given composition, such as the emulsified composition or compositions obtained or derived therefrom. In various versions of the invention at least 80%, at least 85%, at least 90%, at least 95%, or at least 99% of the total number lipid particles in a given composition, such as the emulsified composition or compositions obtained or derived therefrom, have a diameter from 5 nm to 500 nm. In any of the foregoing embodiments, the lipid particles of the invention can have any one of the aforementioned sizes or size ranges for a period of at least 10 days, at least 20, days, at least 30 days, at least 40 days, at least 50 days, at least 60 days, at least 70 days, at least 80 days, at least 90 days, at least 100 days, at least 120, days, at least 130 days, at least 140 days, at least 150 days, at least 160 days, at least 170 days, at least 180 days, at least 190 days, at least 200 days or more. In any of the foregoing embodiments, the lipid particles of the invention can have any one of the aforementioned sizes or size ranges for a period up to 50 days, up to 60 days, up to 70 days, up to 80 days, up to 90 days, up to 100 days, up to 120, days, up to 130 days, up to 140 days, up to 150 days, up to 160 days, up to 170 days, up to 180 days, up to 190 days, up to 200 days or more.

In addition to the step of emulsifying the combined composition, the methods of generating the lipid particles can further comprise a step of size-filtering the lipid particles. The lipid particles can be size-filtered by size-filtering any composition comprising the lipid particles, for example, the emulsified composition and/or the autoclaved composition (as described below), to thereby generate a size-filtered composition. The lipid particles can be filtered using a filter with a pore size from 0.5 μm to 5.0 μm or more, such as from 0.5 μm to 2 μm, or about 1 μm.

The method of generating the lipid particles can further comprise a step of autoclaving the lipid particles. The lipid particles can be autoclaved in any composition comprising the lipid particles, including the emulsified composition and/or the size-filtered composition, to thereby generate an autoclaved composition.

The method of any prior claim, further comprising autoclaving the lipid particles. Methods of autoclaving are well known in the art. Exemplary autoclaving conditions include exposure to pressurized saturated steam at about 121° C. (250° F.) for around 30-60 minutes at a pressure of about 15 psi above atmospheric pressure (205 kPa or 2.02 atm). Variations of these conditions are well known in the art.

In some versions of the invention, the lipid composition used for generating the combined composition is a milk fat composition comprising target milk fat that is generated from a prior milk fat composition. The prior milk fat composition can comprise the target milk fat and additional milk fat. The milk fat composition in such embodiments can be made by removing the additional fat in the prior milk fat composition from the target milk fat to thereby generate the milk fat composition. The removing the additional fat can be performed by any of a variety of lipid purification methods. In some embodiments, the removing the additional fat can comprise melt fractionation. Melt fractionation is described in further detail herein. Briefly, melt fractionating comprises holding a lipid composition at a specific temperature to generate a liquid phase and a solid phase, separating the liquid phase from the solid phase, and retaining either the removed liquid phase or the solid phase depending on the target lipids that are desired. In various versions of the invention, the target milk fat obtained from melt fractionation can comprise a liquid fraction of the prior milk fat composition from a temperature of 0° C., 5° C., 10° C., 15° C., 20° C., 25° C., 30° C., 35° C., 40° C., 45° C., 50° C., 55° C., 60° C., 65° C., 70° C., 75° C., 80° C., 85° C., or any range between and including any two of the foregoing values. In various versions of the invention, the target milk fat obtained from melt fractionation can comprise a solid fraction of the prior milk fat composition from a temperature of 0° C., 5° C., 10° C., 15° C., 20° C., 25° C., 30° C., 35° C., 40° C., 45° C., 50° C., 55° C., 60° C., 65° C., 70° C., 75° C., 80° C., 85° C., or any range between and including any two of the foregoing values. In preferred embodiments, the target milk fat obtained from melt fractionation can comprise a liquid fraction of the prior milk fat composition from a temperature of from 15° C. to 35° C., such as from 20° C. to 30° C., or about 25° C.

The prior milk fat composition can comprise milk or any downstream product derived therefrom. The prior milk fat composition preferably comprises a composition generated from milk by removing at least a portion of solids-not-fat and/or water from the milk. In various versions of the invention, the prior milk fat composition can comprise any of the characteristics described above for the lipid composition, including but not limited to any of the above-referenced total lipid amounts, any of the above-referenced milk fat amounts (e.g., in a solids portion thereof), and any of the above-referenced solids portion amounts. Exemplary prior milk fat compositions comprise anhydrous milk fat, butter oil, and ghee, among others.

The lipid particles of the invention can be used for parenteral nutrition. Accordingly, some versions of the invention are directed to methods of administering parenteral nutrition. The methods can comprise parenterally administering the lipid particles of the invention to a subject. The subject can be a subject in need of parenteral nutrition. The lipid particles of the invention have reduced deleterious immunological effects than intravenous fat emulsions made from vegetable fats conventionally used in parenteral nutrition. The particles of the invention can therefore be used for parenteral nutrition for subjects with various inflammatory complications or disorders, such as pancreatitis, hepatitis, or other inflammatory complications.

The lipid particles of the invention can be administered in the form of a parenteral nutrition composition. The parenteral nutrition composition can comprise the particles of the invention in a carrier. Parenteral nutrition carriers are well known in the art.

The particles of the invention can be administered separately from other parenteral nutrition solutions or in all-in-one parenteral nutrition compositions containing the lipid particles of the invention in combination with other nutrients. Compositions containing all essential nutrients are preferred to reduce the possibility of microbiological contamination, simplify in-home usage, decrease nursing time, and decrease risk of error (Hardy and Puzovic, 2009). Exemplary parenteral nutrition nutrients include amino acids and dextrose, among others. “Intralipid®” is a registered trademark of Riker Laboratories, Inc., Northridge, CA.

Intralipid® 20% (a 20% intravenous fat emulsion) is a sterile, non-pyrogenic fat emulsion prepared for intravenous administration as a source of calories and essential fatty acids. It is made up of 20% soybean oil, 1.2% egg yolk phospholipids, 2.25% glycerin, and water for injection. In addition, sodium hydroxide has been added to adjust the pH so that the final product pH is 8. pH range is 6 to 8.9. Intralipid® 20% can be obtained by Baxter Healthcare Corporation, Deerfield, IL.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Overview

### IVFEs and Parenteral Nutrition

For parenteral nutrition, intravenous fat emulsions (IVFEs) containing suspended fat droplets (200-500 nm in diameter) can be prepared using an emulsifying agent (e.g., egg lecithin). IVFEs can be prepared in a manner to avoid essential FA deficiency and other complications (Anez-Bustillos et al., 2016). Essential FA deficiency occurs when <1-2% of energy consumed comes from essential FA (i.e., α-linolenic acid and linoleic acid). This is far more common in patients who are dependent on IVFEs than the general population. Patients who rely on IVFEs can also experience fat overload syndrome (i.e., elevated plasma triglycerides), hepatic steatosis (i.e., fatty liver disease), and other complications, reflecting the importance of balanced FA compositions when utilizing IVFEs for patients requiring parenteral nutrition. Here, we show that milk lipids will have a broad FA profile that can be manipulated and be prepared as a solution to remedy this important clinical problem.

### Milk Fat Composition and Structure

Milk fat is ca. 95-98% triglycerides with 0.8-1.1% phospholipids and minimal (<0.5%) monoglycerides and free FAs (Kailasapathy, 2015). The FA profile of milk is diverse with more than 400 distinct FAs detected. On average, milk fat contains ca. 62% saturated FAs, 29% monounsaturated FAs, and 4% polyunsaturated FAs with palmitic acid (16:0, ca. 29.5% of the FAs in milk) and oleic acid (18:1, ca. 27.4% of the FAs in milk) being the most abundant FAs (Table 1). Additionally, compared to other fat sources, milk fat is relatively abundant in short chain FAs (C₄-C₈), conjugated linoleic acid, and contains detectable essential FAs depending on the feeds used. With every FA having a characteristic melting temperature, the diverse FA profile of milk is responsible for the uniquely broad melting profile in dairy products (e.g., butter).

TABLE 1
Fatty acid profile (expressed in % composition) of bovine milk fat without fractionation
(control) or post-fractionation as well as commercially available lipid emulsions
used in parenteral nutrition, including: Intralipid ® 20% and SMOFlipid ®.

| Fatty | | | | | |
|---|---|---|---|---|---|
| Acid | Common Name | Control | LMF-10 | Intralipid ® | SMOFlipid ® |
| 4:0 | Butyric acid | 3.55 ± 0.06 | 4.63 ± 0.15 | NS | NS |
| 6:0 | Caproic acid | 2.24 ± 0.04 | 2.75 ± 0.13 | NS | NS |
| 8:0 | Caprylic acid | 1.27 ± 0.11 | 1.84 ± 0.06 | NS | 13-24 |
| 10:0 | Capric acid | 3.14 ± 0.06 | 4.27 ± 0.09 | NS | 5-15 |
| 12:0 | Lauric acid | 4.28 ± 0.04 | 5.09 ± 0.08 | NS | NS |
| 14:0 | Myristic acid | 11.92 ± 0.33 | 12.51 ± 0.17 | NS | NS |
| 16:0 | Palmitic acid | 30.55 ± 1.55 | 18.46 ± 0.19 | 7-14 | 7-12 |
| 18:0 | Stearic acid | 10.55 ± 0.72 | 5.19 ± 0.05 | 1.4-5.5 | 1.5-4 |
| 18:1 | Oleic acid | 23.19 ± 0.44 | 31.12 ± 0.71 | 19-30 | 23-35 |
| 18:2 | Linoleic acid | 1.88 ± 0.13 | 3.16 ± 0.09 | 44-62 | 14-25 |
| 18:3 | Linolenic acid | NS | — | 4-11 | 1.5-3.5 |
| 20:5 | Eicosapentaenoic | NS/SD | — | — | 1-3.5 |
| | acid (EPA) | | | | |
| 22:6 | Docosahexaenoic | NS | — | — | 1-3.5 |
| | acid (DHA) | | | | |

LMF-10 = low-melting fractions obtained at 10° C.; Intralipid ® lipids = 100% soybean oil; SMOFlipid ® lipids = 30% soybean oil, 30% coconut oil, 25% olive oil, and 15% fish oil; NS = not specified.
SD = sometimes detected.

Milk FA profile can be altered by direct manufacturing interventions such as melt fractionation, as outlined herein. Typically, milk fat fractionation takes advantage of the broad melting range of milk fat, allowing for the controlled crystallization and removal of specific groups of FAs. For example, milk fat can be fractionated by incubating cream at a specific temperature, allowing some fat to crystallize, and then separating the liquid fat from the fat crystals via filtration, centrifugation, or any other suitable method, such as methods suitable for phase separation. The present examples employ melt curve to generate favorable FA profiles for parenteral nutrition.

The fat content and FA profile of milk can also be manipulated during milk production through cow genetics (e.g., breed; Coffey et al., 2016), lactation and milking stages (Rico et al., 2014), diet (Atkins et al., 2020), and environmental factors (e.g., season; Bailey et al., 2005). For example, many studies have found that α-Linolenic acid (18:3) content in milk increases in pasture-fed cows compared to cows fed a total mixed ration ad libitum (Barca et al., 2017, Atkins et al., 2020).

Concentrated milkfat sources are preferred lipid sources for prepare the exemplary IVFE of the invention. These include anhydrous milkfat and butter oil, among others (Table 2). These lipid sources preferably have at least some of the solids-not-fat constituents of milk removed.

TABLE 2
Composition of concentrated milkfat sources

| Constituent | Butter | Butteroil | Anhydrous milkfat |
|---|---|---|---|
| Milkfat | ≥80.0% | ≥99.6% | ≥99.8% |
| Moisture | ND | ≤0.3% | ≤0.1% |
| Other butter | ND | ≤0.1% | ≤0.1% |
| constituents | | | |
| Free fatty acids | ND | ≤0.5% (calculated | ≤0.3% (calculated |
| | | as oleic acid) | as oleic acid) |

ND = not defined

### Ultra High-Pressure Homogenization for Micelle Formation

In milk, lipids are primarily contained in globules (1-20 μm in diameter) surrounded by a milk fat globule membrane (MFGM, 5-10 nm thick; Kailasapathy, 2015). The native MFGM, containing proteins, polar lipids, lipoproteins, phospholipids, and other components, helps to protect milk fat from lipoprotein lipase (i.e., reducing the rate of hydrolytic rancidity) and stabilizes milk fat in an emulsion (i.e., reducing the rate of creaming). Conventional homogenization (ca. 10-20 MPa), which involves forcing liquid product through flow restriction(s), effectively splits the milk fat globules into many smaller globules (average size <1 μm) and subsequently disrupts the native MFGM (Chandan, 2015).

Ultra high-pressure homogenization (UHPH) operates under more extreme pressures (100-400 MPa) than conventional dairy homogenization using abrasion-resistant nozzles, inducing more extreme shear, friction, cavitation, and heat to samples. This processing methodology has been shown to alter protein quaternary structures (Harte et al., 2002), change polysaccharide functionality (Harte and Venegas, 2010), inactivate microorganisms (Diels and Michiels, 2006), and enhance emulsion stability (Galvão et al., 2018). UHPH induces the formation of small, monodisperse droplets, especially when droplet re-coalescence is discouraged by adding surfactants and optimizing processing pressure to reduce UHPH-induced heating and decrease cavitation. UHPH is employed in the present examples to achieve IVFE sizes similar to circulating chylomicrons for use in parenteral nutrition.

### Dairy Solution for Intravenous Lipid Emulsions

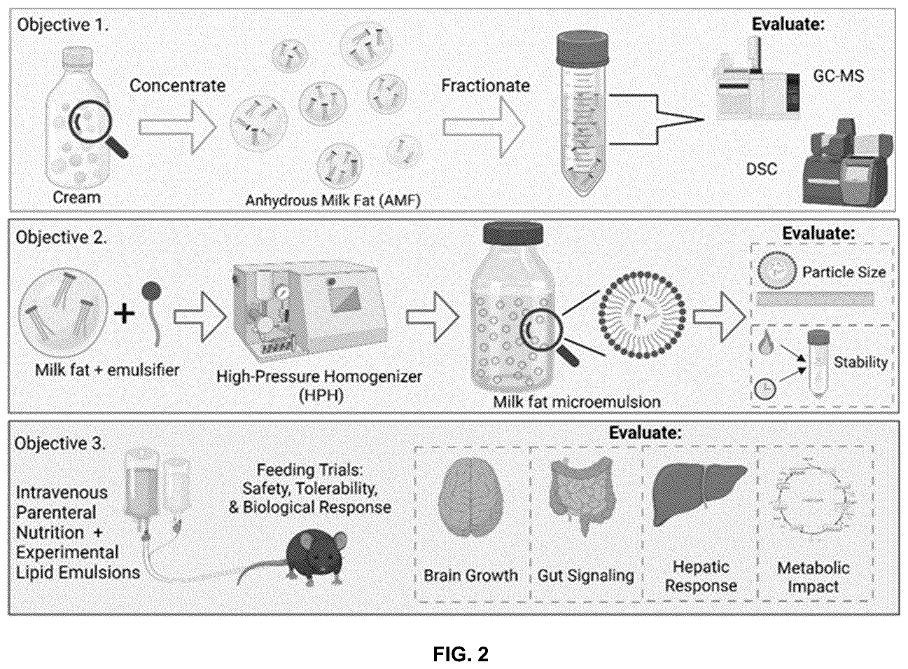

We show herein that extracting and purifying bovine milk lipids is a solution to the supply chain and lipid sourcing space that convers unique metabolic and immunological health benefits in patients requiring intravenous nutrition. We also show that extracting and purifying bovine milk lipids is a solution to the supply chain and lipid sourcing space that confers unique metabolic and immunological health benefits in patients requiring intravenous nutrition. The objectives of the present examples are to: (1) Melt fractionate an initial milk fat composition into a preferrable FA profile; (2) Develop a stable milk fat emulsion suitable for intravenous usage using UHPH; and (3) Evaluate the suitability of emulsified milk fat and identified milk fat fractions suitable for intravenous lipid support (FIG. 2 and Table 3).

TABLE 3
The objectives, hypotheses and, and deliverables with this work.

| Objectives | Hypotheses | Deliverables |
|---|---|---|
| Objective 1. Develop a | | Improved fatty acid profile for |
| desirable fatty acid | | IVFEs. |
| profile using | | |
| fractionation by | | |
| monitoring fatty acid | | |
| composition using GC-MS. | | |
| Objective 2. Evaluate the | A UHPH pressure of 150 MPa | Optimized procedure for milk |
| particle size and stability | (0.20 mm orifice) with some | fat-based IVFE preparation. |
| of milk fat-based IVFEs | back pressure will be sufficient | |
| made using high-pressure | to produce IVFEs suitable for | |
| homogenization with | intravenous use (<500 nm). | |
| varied processing | Droplet size will decrease and | |
| parameters. | stability will increase with | |
| | higher processing pressures if | |
| | coalescence can be controlled. | |
| Objective 3. Determine | Purified milk fat lipid fraction | Milk fat-based IVFEs suitable |
| the suitability of milk fat- | emulsions will support | for parenteral nutrition. |
| based IVFEs for | metabolism and optimize | |
| intravenous lipid support | organ growth and function | |
| using mouse models. | throughout the lifespan. | |

### Objective 1. Develop a Desirable Fatty Acid Profile Using Fractionation by Monitoring Fatty Acid Composition Using GC-MS.

We identify herein suitable milk fat sources (e.g., anhydrous milk fat), fractionate milk fat using controlled crystallization, and evaluate components using GCMS+DSC (crystallization).

Objective 2. Evaluate the Particle Size and Stability of Milk Fat-Based IVFEs Made Using High-Pressure Homogenization with Varied Processing Parameters.

Beyond selecting a FA profile that is suitable for parenteral nutrition, other issues associated with IVFEs are the desire to (1) generate lipid particles that are very small (200-500 nm), (2) generate lipid particles that are stable to typical handling and storage practices, and (3) avoid inadvertently introducing microorganisms that increase the risk of infection (Hardy and Puzovic, 2009). A UHPH system is used for generating the lipid particles (Nano DeBEE 45-2, BEE International) is used to produce highly stable particles with adequate diameters suitable for IVFEs. Multiple emulsifiers and UHPH processing parameters will be utilized to optimize the particle size, storage stability, and heat stability of IVFEs (Table 4).

TABLE 4
The variables to be tested in IVFE formation,
including emulsifier and HPH parameters.

| Parameter | Variables |
|---|---|
| Emulsifier | Type - lecithin, glycerin |
| | Concentration - 0.5%, 1%, 1.5% |
| | Surfactant-to-lipid ratio - 1:4, 1:3, 1:2 |
| UHPH conditions | Pressure - 150 MPa, 300 MPa |
| | Back pressure - 0 psi, 1000 psi, 2000 psi |
| | Orifice size - 0.13 mm, 0.20 mm |
| | Number of passes - 1, 2, or 3 |
| | Other options: Parallel or reverse flow, reactor cell |
| | orientation, input/output temperature |

IVFE particle size can be determined using a Malvern MasterSizer 3000 (at UW-Madison), as described in Shi et al. (2009). IVFE storage stability can be determined by monitoring IVFE particle size at 15 day intervals through 12 months of storage at 4° C. and 20° C. Heat stability can be evaluated by holding prepared IVFEs in water baths at 40, 60, 80, and 90° C. and collecting aliquots at 0, 1, 2, 4, 8, 12, 24, and 48 h for particle size determination. IVFE microbiological safety can be determined following sterile filtration methods followed by endotoxin testing, 16S amplification, and aerobic and anerobic culturing techniques to confirm the presence or absence of viable or non-viable microorganisms remain in emulsion solutions. Additional processing steps can be introduced to promote sterility, including (1) a 0.2 μm filtration step or (2) heat sterilization prior.

### Objective 3. To Determine the Suitability of Milk Fat-Based IVFEs for Intravenous Lipid Support Using Mouse Models.

The dairy based IVFEs can be tested in preclinical animal models alongside existing commercial lipid sources, including Intralipid® and SMOFlipid®. Following intravenous catheterization under sterile technique, animals can be fed parenteral nutrition and experimental lipid emulsions at 5-10% volume/volume. Administration rates can be based on animal body weight, tolerability, and calculated to meet caloric, nitrogen, and fat requirements for the animals. Systemic lipid metabolism, including clearance and uptake, can be examined. After a period of time (e.g., 5 days), animals can be humanely sacrificed for additional metabolic and immunological responses. Solutions can first be tested in adult animals (6-8 weeks of age) to establish safety, tolerability, and baseline metabolic impacts between the lipid emulsion formulations. Following this, pediatric animals (8-10 days of age) can be used, since mice at this age are developmentally equivalent to human newborns who often require parenteral nutrition. Animals can be fed for a period of time (e.g., 5 days) prior to assessment of hepatic homeostasis, endocrine signaling, organ development and body weight gain. In both sets of experiments, adult control animals can receive a jugular catheter and be provided saline with ab libitum access to food, or pediatric animals can undergo jugular vein occlusion to control for the presence of a catheter and be returned to the dam to continue maternal milk feeding.

### Materials, Methods, and Results

### Fat Fractionation

Milk fat with high purity was obtained in the form of anhydrous milkfat (AMF, Grassland, Greenwood, WI).

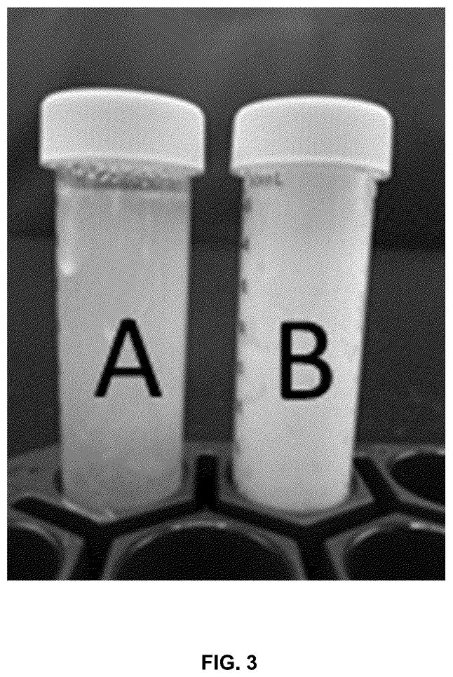

Native AMF was fractionated using dry fractionation at fractionation temperatures of 25 and 15° C. Specifically, the AMF was heated to >65° C. (e.g., 80° C.) to erase any crystal nuclei memory (i.e., melted), then the AMF was slowly cooled to the desired fractionation temperature and held at this temperature for 24 h under constant agitation in a rotary evaporator (Rotavapor R-100, BUCHI Corporation, New Castle, DE). The solid and liquid fractions were separated using vacuum filtration. Centrifugation or other suitable methods, such as methods for phase separation, can be used. Samples of the unfractionated AMF as well as high melting fractions (HMFs, solid portion at each respective temperature) and low melting fractions (LMFs, liquid portion at respective temperature) at each temperature were collected and analyzed (FIG. 3). The fractionated samples were designated as: HMF-25, LMF-25, HMF-15, LMF-15.

### Lipid Analysis

### Lipid Sample Preparation

Milk fat fractions were stored in 1.5 mL centrifuge tubes. Samples were either solid or liquid at room temperature, therefore, in order to render the samples suitable for manipulation, all samples were heated to 37° C. in a water bath. Samples that remained solid at 37° C. were heated further until fully liquid. All samples were diluted 25-fold (25×) in isopropyl alcohol (IPA) by adding 20-μL sample to 480 μL IPA and vortexing. These 25× dilutions were then held at 37° C. to keep solids from forming while the samples underwent further dilution. For positive mode analysis, samples were diluted a further 40× with IPA to a final dilution factor of 1000×. For negative mode analysis, samples were diluted to a final dilution factor of 100× with IPA. These dilution factors were chosen to both yield a sufficient signal level in positive ion mode, while attempting to prevent excessive overloading of the chromatographic system and generating carryover. The negative ion dilution factors were chosen with the knowledge that the material detected in positive ion mode is being loaded onto the chromatographic and mass spectrometric systems, even though the lipids giving rise to those signals are not visible in negative ion mode. After preparing all dilution levels, the diluted samples were placed at 4° C. for approximately 15 minutes to determine if any solids would precipitate, because this is the temperature that the autosampler is maintained at while samples are waiting to be run. No solids were observed.

### Lipid Data Acquisition

Samples were analyzed by ultra-high-performance liquid chromatography/mass spectrometry (UHPLC/MS) and ultra-high-performance liquid chromatography/mass spectrometry/mass spectrometry (UHPLC/MS/MS) in positive ion and negative ion modes. The UHPLC conditions were the same for all acquisitions, regardless of ionization polarity, dilution factor, or MS level (MS or MS/MS). The solvents consisted of A: 10 mM ammonium formate, 0.1% (v/v) formic acid, 60% (v/v) acetonitrile in water; and B: 10 mM ammonium formate, 0.1% (v/v) formic acid, 9% (v/v) acetonitrile, 1% (v/v) water in 2-propanol. The column was a Waters Acquity UPLC BEH C18 1.7 μm 2.1 mm×100 mm, with a guard column containing the same stationary phase with dimensions 2.1 mm×5 mm. The gradient is shown in Table 5 below.

TABLE 5

| | | | |
|---|---|---|---|
| Time | % A | % B | Flow |

| | | | | |
|---|---|---|---|---|
| 0.00 | min | 85.00% | 15.00% | 0.500 mL/min |
| 2.40 | min | 70.00% | 30.00% | 0.500 mL/min |
| 3.00 | min | 52.00% | 48.00% | 0.500 mL/min |
| 13.20 | min | 18.00% | 82.00% | 0.500 mL/min |
| 13.80 | min | 1.00% | 99.00% | 0.500 mL/min |
| 15.40 | min | 1.00% | 99.00% | 0.500 mL/min |
| 16.00 | min | 85.00% | 15.00% | 0.500 mL/min |
| 20.00 | min | 85.00% | 15.00% | 0.500 mL/min |

The column was maintained at 50° C. Samples were placed in an autosampler held at 8° C. until injection. The UHPLC was an Agilent model 1290 Infinity II with individual components consisting of a model G7120A binary pump, model G7167B multisampler, model G7116B column compartment, and model G7110B isocratic pump. The HPLC was connected to the inlet port of an Agilent G6546A QTOF mass spectrometer, incorporating an Agilent JetStream dual ESI source. The column effluent was delivered to the sample nebulizer of the dual ESI source, while the isocratic pump delivered internal calibrant to the reference nebulizer of the dual ESI source. QTOF parameters differed depending on the ionization polarity and MS level of the acquired data. Parameters for individual acquisition methods are shown below.

Sample injection volumes also varied depending on MS level and polarity. For positive ion MS, injection volumes were 2 μL; for negative ion MS, injection volumes were 5 μL; for positive ion MS/MS, injection volumes were 4 μL; for negative ion MS/MS injection volumes were 7 μL. After sample pickup, the needle was washed for 3s in the autosampler flush port with 1:1 2-propanol:methanol. LC/MS data were collected with one technical replicate injection per sample. LC/MS/MS data were collected in iterative mode as described above with 5 iterative injections made per ionization mode.

### Lipid Data Analysis

Assignment of lipid identities to mass and retention time signal pairs was made using Lipid Annotator software (Agilent) (Koelmel et al, Metabolites. 10 (3); 101. 2020) and the LC/MS/MS data. Lipid Annotator uses the accurate mass of the precursor and product ions observed within the fragmentation spectra to assign a dominant constituent lipid to a molecular feature in the data, or where that is not possible, a sum composition. Dominant compositions have lipid acyl chains and degree of unsaturation explicitly enumerated, whereas sum compositions only indicate the lipid class, total carbon number, and number of unsaturated sites. After assignment of lipid identities by Lipid Annotator, a database is exported containing the lipid, the mass, and the retention time. This database can then be used by Profinder software (Agilent) to align retention times across samples and extract and integrate ion chromatograms for each lipid in each sample LC/MS data file. These integrations are then reviewed for accuracy and a comma-separated value (.csv) file exported for further analysis. Because lipids are frequently present in different structural isomers, which can be resolved chromatographically, there are often more than a single chromatographic peak assigned to the same lipid identity. This is particularly true in cases where only a sum composition can be reported, and the acyl chains cannot be individually identified. In these cases, the exported.csv file will have multiple entries assigned to the same lipid species, so a suffix “#” is added to the lipid i.d. to distinguish the different chromatographic species.

Because the goal of the current experiment was to identify the lipids present in the various fractions and evaluate the fatty acid profile, peak integrations were not performed and data were evaluated by comparing the lipid identifications from Lipid Annotator.

### Lipid Findings

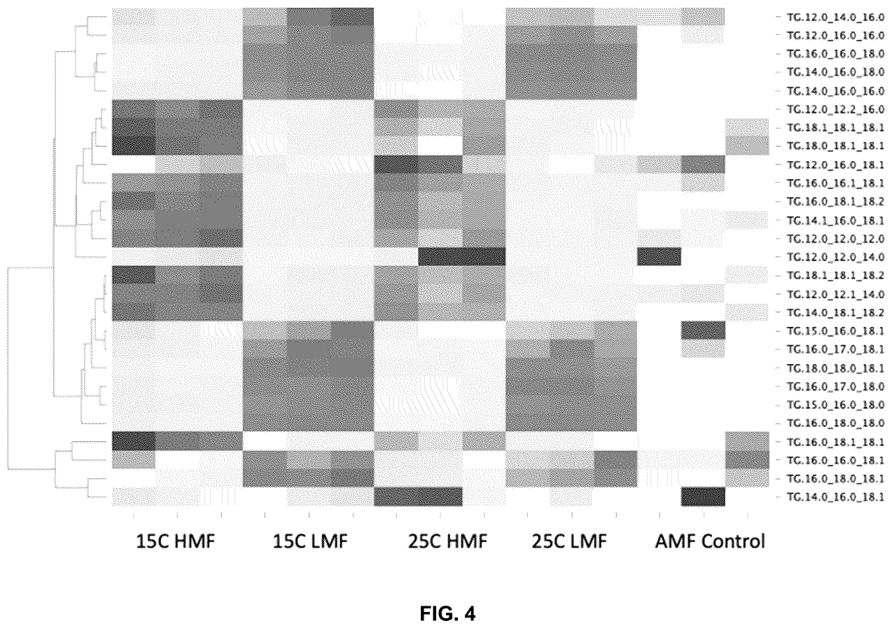

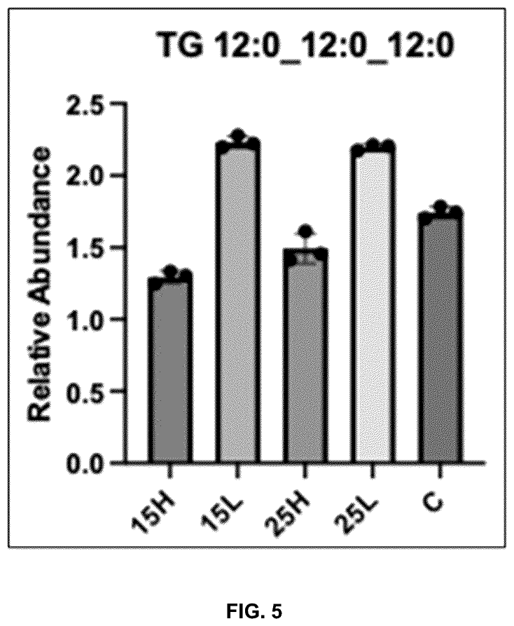

The relative abundance of triglycerides are shown in FIG. 4 for AMF, HMF-15 (15° C. high melting fraction), LMF-15 (15° C. low melting fraction), HMF-25 (25° C. high melting fraction), and LMF-25 (25° C. low melting fraction). Raw mass spec data obtained on identified lipid targets obtained by liquid chromatography with tandem mass spectrometry are provided in Tables 6A-6F. The triglycerides with three medium-chain, saturated (12:0) fatty acids (lauric acid) esterified to the glycerol backbone are shown in FIG. 5 for was compared among the AMF (“C” in FIG. 5), HMF-15 (“15H” in FIG. 5), LMF-15 (“15L” in FIG. 5), HMF-25 (“25H” in FIG. 5), and LMF-25 (“25L” in FIG. 5). These medium-chain triglycerides were abundant in the LMFs and are associated with numerous health benefits (Jadhav & Annapure, 2023).

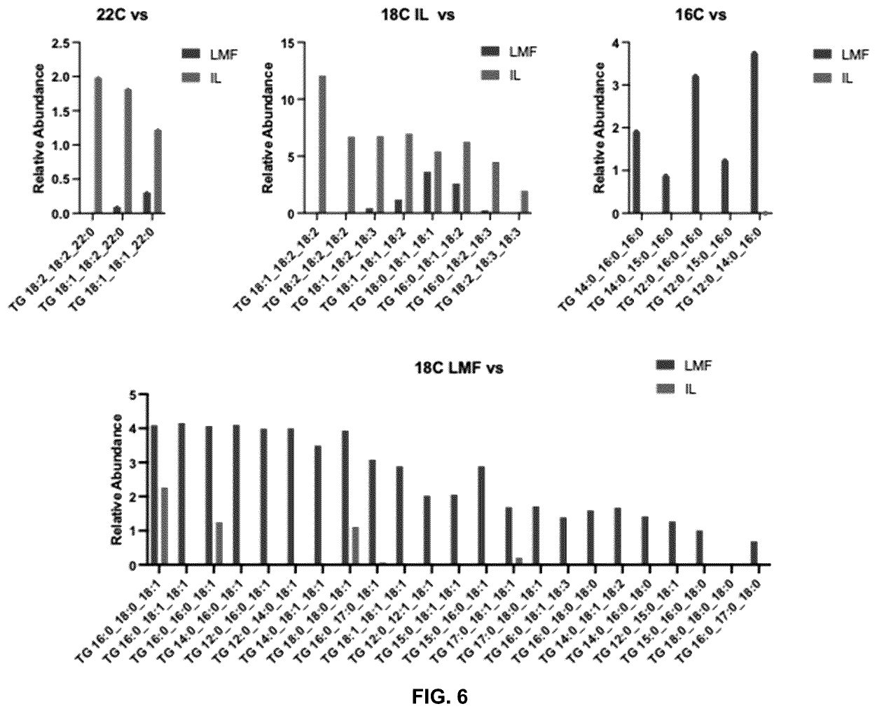

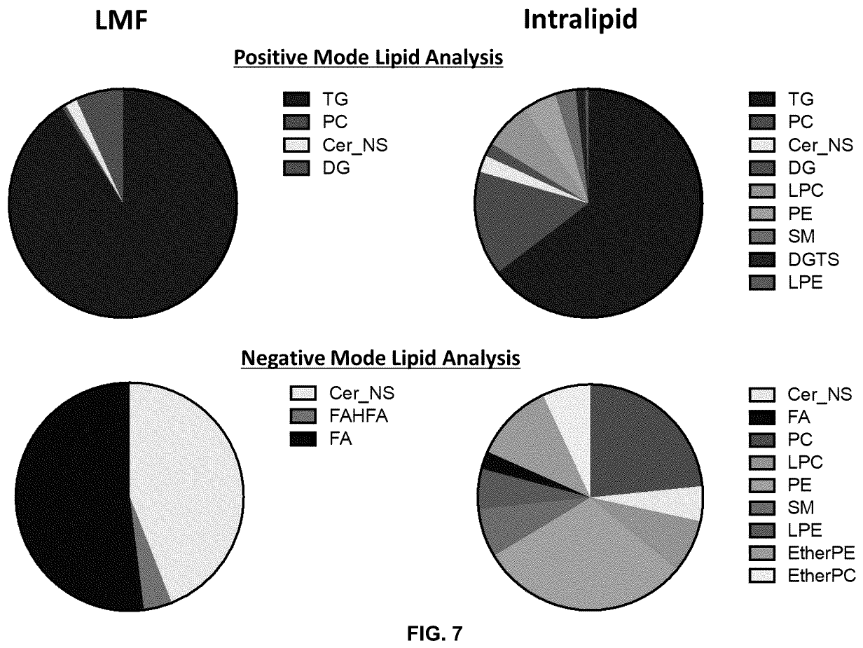

The lipids in LMF-25 and a conventional soy protein intravenous fat emulsion (Intralipid® 20%) were analyzed and compared (FIGS. 6 and 7). LMF-25 had a much more diverse fatty acid profile compared to Intralipid® 20%. LMF-25 showed some 22-carbon triglyceride acyl groups but was enriched in 12-18-carbon triglyceride acyl groups. By contrast, Intralipid® 20% was dominated by 18- and 22-carbon triglyceride acyl groups.

TABLE 6A
Raw mass spec data obtained on identified lipid targets obtained
by liquid chromatography with tandem mass spectrometry:
Formula, mass and retention time (rt) of compounds.

| Compound Name | Formula | Mass | RT |
|---|---|---|---|
| DG 16:0_16:0 | C35 H68 O5 | 568.5066 | 9.91 |
| DG 18:0_18:1 | C39 H74 O5 | 622.5536 | 10.77 |
| PC 18:1_19:1 | C45 H86 N O8 P | 799.6091 | 9.01 |
| PC 34:1 | C42 H82 N O8 P | 759.5778 | 8.38 |
| PC 36:2 | C44 H84 N O8 P | 785.5933 | 8.36 |
| PC 37:2 | C45 H86 N O8 P | 799.609 | 8.8 |
| TG 12:0_12:0_12:0 | C39 H74 O6 | 638.5489 | 10.94 |
| TG 12:0_12:0_12:1 | C39 H72 O6 | 636.5329 | 10.11 |
| TG 12:0_12:0_13:0 | C40 H76 O6 | 652.5642 | 11.33 |
| TG 12:0_12:0_14:0 | C41 H78 O6 | 666.5804 | 11.71 |
| TG 12:0_12:0_15:0 | C42 H80 O6 | 680.5955 | 11.95 |
| TG 12:0_12:0_17:0 | C44 H84 O6 | 708.6267 | 12.62 |
| TG 12:0_12:0_17:1 | C44 H82 O6 | 706.6112 | 11.96 |
| TG 12:0_12:1_14:0 | C41 H76 O6 | 664.5644 | 10.94 |
| TG 12:0_12:1_14:3 | C41 H70 O6 | 658.5174 | 8.88 |
| TG 12:0_12:1_20:5 | C47 H78 O6 | 738.5801 | 10.13 |
| TG 12:0_12:2_15:0 | C42 H76 O6 | 676.5641 | 10.71 |
| TG 12:0_12:2_16:0 | C43 H78 O6 | 690.5801 | 11.08 |
| TG 12:0_12:2_18:2 | C45 H78 O6 | 714.5797 | 10.46 |
| TG 12:0_14:0_16:0 | C45 H86 O6 | 722.643 | 12.94 |
| TG 12:0_14:0_20:4 | C49 H86 O6 | 770.6417 | 12.15 |
| TG 12:0_15:0_16:0 | C46 H88 O6 | 736.658 | 13.24 |
| TG 12:0_15:0_16:0 | C46 H88 O6 | 736.6579 | 13.12 |
| TG 12:0_16:0_16:0 | C47 H90 O6 | 750.6743 | 13.53 |
| TG 12:0_16:0_16:0 | C47 H90 O6 | 750.6733 | 14.45 |
| TG 12:0_16:0_18:1 | C49 H92 O6 | 776.6895 | 13.51 |
| TG 13:0_18:1_18:2 | C52 H94 O6 | 814.7053 | 13.27 |
| TG 14:0_14:0_16:0 | C47 H90 O6 | 750.674 | 13.25 |
| TG 14:0_14:3_16:0 | C47 H84 O6 | 744.6253 | 12.48 |
| TG 14:0_14:3_18:0 | C49 H88 O6 | 772.655 | 13.1 |
| TG 14:0_15:0_16:0 | C48 H92 O6 | 764.6893 | 13.8 |
| TG 14:0_15:0_16:0 | C48 H92 O6 | 764.6892 | 13.69 |
| TG 14:0_15:0_18:1 | C50 H94 O6 | 790.7049 | 13.78 |
| TG 14:0_16:0_16:0 | C49 H94 O6 | 778.7057 | 14.07 |
| TG 14:0_16:0_17:1 | C50 H94 O6 | 790.7049 | 14.18 |
| TG 14:0_16:0_18:0 | C51 H98 O6 | 806.7371 | 14.37 |
| TG 14:0_16:0_18:1 | C51 H96 O6 | 804.7209 | 14.05 |
| TG 14:0_16:0_18:1 | C51 H96 O6 | 804.7197 | 14.59 |
| TG 14:0_16:0_20:4 | C53 H94 O6 | 826.7043 | 13.38 |
| TG 14:0_16:0_20:5 | C53 H92 O6 | 824.6887 | 12.94 |
| TG 14:0_18:1_18:2 | C53 H96 O6 | 828.7207 | 13.54 |
| TG 14:1_16:0_18:1 | C51 H94 O6 | 802.7054 | 13.51 |
| TG 15:0_16:0_18:0 | C52 H100 O6 | 820.752 | 14.43 |
| TG 15:0_16:0_18:1 | C52 H98 O6 | 818.7362 | 14.23 |
| TG 16:0_16:0_17:1 | C52 H98 O6 | 818.7361 | 14.42 |
| TG 16:0_16:0_18:0 | C53 H102 O6 | 834.7683 | 14.53 |
| TG 16:0_16:0_18:1 | C53 H100 O6 | 832.7526 | 14.36 |
| TG 16:0_16:0_20:4 | C55 H98 O6 | 854.7355 | 13.92 |
| TG 16:0_16:1_18:1 | C53 H98 O6 | 830.7369 | 14.04 |
| TG 16:0_17:0_18:0 | C54 H104 O6 | 848.7832 | 14.57 |
| TG 16:0_17:0_18:1 | C54 H102 O6 | 846.7675 | 14.44 |
| TG 16:0_17:1_18:0 | C54 H102 O6 | 846.767 | 14.57 |
| TG 16:0_18:0_18:0 | C55 H106 O6 | 862.7993 | 14.63 |
| TG 16:0_18:0_18:1 | C55 H104 O6 | 860.7834 | 14.52 |
| TG 16:0_18:0_21:0 | C58 H112 O6 | 904.8456 | 14.74 |
| TG 16:0_18:1_18:1 | C55 H102 O6 | 858.7678 | 14.35 |
| TG 16:0_18:1_18:2 | C55 H100 O6 | 856.7524 | 14.07 |
| TG 16:0_18:1_18:3 | C55 H98 O6 | 854.7362 | 13.63 |
| TG 16:0_18:1_20:5 | C57 H98 O6 | 878.7357 | 13.49 |
| TG 16:0_20:0_22:0 | C61 H118 O6 | 946.8926 | 14.85 |
| TG 17:0_18:0_18:0 | C56 H108 O6 | 876.8141 | 14.66 |
| TG 17:0_18:0_18:1 | C56 H106 O6 | 874.7988 | 14.57 |
| TG 17:0_18:1_18:1 | C56 H104 O6 | 872.7832 | 14.44 |
| TG 17:1_18:0_18:1 | C56 H104 O6 | 872.7828 | 14.56 |
| TG 18:0_18:0_18:0 | C57 H110 O6 | 890.8299 | 14.71 |
| TG 18:0_18:0_18:1 | C57 H108 O6 | 888.815 | 14.62 |
| TG 18:0_18:1_18:1 | C57 H106 O6 | 886.7999 | 14.51 |
| TG 18:1_18:1_18:1 | C57 H104 O6 | 884.7843 | 14.34 |
| TG 18:1_18:1_18:2 | C57 H102 O6 | 882.7678 | 14.06 |
| TG 18:1_18:2_18:2 | C57 H100 O6 | 880.7519 | 13.61 |
| TG 18:1_18:2_18:3 | C57 H98 O6 | 878.7361 | 13.14 |
| TG 18:1_18:3_18:3 | C57 H96 O6 | 876.7208 | 12.66 |
| TG 18:1_20:0_22:0 | C63 H120 O6 | 972.9082 | 14.85 |
| TG 38:2 | C41 H74 O6 | 662.5485 | 10.24 |
| TG 38:3 | C41 H72 O6 | 660.5327 | 9.57 |
| TG 40:0 | C43 H82 O6 | 694.6116 | 12.3 |
| TG 40:1 | C43 H80 O6 | 692.5956 | 11.71 |
| TG 40:3 | C43 H76 O6 | 688.5642 | 10.4 |
| TG 40:4 | C43 H74 O6 | 686.5485 | 9.76 |
| TG 42:1 | C45 H84 O6 | 720.6271 | 12.3 |
| TG 42:4 | C45 H78 O6 | 714.5798 | 10.7 |
| TG 43:1 | C46 H86 O6 | 734.6424 | 12.62 |
| TG 44:1 | C47 H88 O6 | 748.6582 | 12.93 |
| TG 44:2 | C47 H86 O6 | 746.6425 | 12.33 |
| TG 44:5 | C47 H80 O6 | 740.5957 | 10.8 |
| TG 45:1 | C48 H90 O6 | 762.6736 | 13.22 |
| TG 46:2 | C49 H90 O6 | 774.6739 | 12.93 |
| TG 46:3 | C49 H88 O6 | 772.658 | 12.36 |
| TG 47:0 | C50 H96 O6 | 792.7206 | 14.24 |
| TG 47:1 | C50 H94 O6 | 790.7049 | 13.78 |
| TG 47:2 | C50 H92 O6 | 788.6893 | 13.22 |
| TG 47:3 | C50 H90 O6 | 786.6739 | 12.69 |
| TG 48:3 | C51 H92 O6 | 800.6893 | 12.98 |
| TG 48:4 | C51 H90 O6 | 798.6731 | 12.79 |
| TG 48:5 | C51 H88 O6 | 796.658 | 12.12 |
| TG 49:2 | C52 H96 O6 | 816.7206 | 13.77 |
| TG 51:2 | C54 H100 O6 | 844.7521 | 14.22 |
| TG 51:4 | C54 H96 O6 | 840.7207 | 13.37 |
| TG 52:5 | C55 H96 O6 | 852.7166 | 13.52 |
| TG 52:5 | C55 H96 O6 | 852.7206 | 13.15 |
| TG 52:6 | C55 H94 O6 | 850.7061 | 12.76 |
| TG 53:4 | C56 H100 O6 | 868.7519 | 13.83 |
| TG 54:5 | C57 H100 O6 | 880.7517 | 13.9 |
| TG 55:1 | C58 H110 O6 | 902.8296 | 14.67 |
| TG 56:1 | C59 H112 O6 | 916.845 | 14.71 |
| TG 56:6 | C59 H102 O6 | 906.7677 | 13.88 |
| TG 57:0 | C60 H116 O6 | 932.8767 | 14.81 |
| TG 57:2 | C60 H112 O6 | 928.8456 | 14.66 |
| TG 58:2 | C61 H114 O6 | 942.8612 | 14.7 |

TABLE 6B
Raw mass spec data obtained on identified lipid targets obtained
by liquid chromatography with tandem mass spectrometry: HMF-15.

| | HMF-15 | HMF-15 | HMF-15 |
|---|---|---|---|
| Compound Name | Replicate 1 | Replicate 2 | Replicate 3 |
| DG 16:0_16:0 | 0.06384539 | 0.066072921 | 0.066587608 |
| DG 18:0_18:1 | 0.021972192 | 0.013111251 | 0.022672526 |
| PC 18:1_19:1 | 0.005104499 | 0.00676606 | 0.008570779 |
| PC 34:1 | 0.006087907 | 0.007937543 | 0.007025569 |
| PC 36:2 | 0.009927778 | 0.013034357 | 0.017802487 |
| PC 37:2 | 0.002975157 | 0.005052031 | 0.005730281 |
| TG 12:0_12:0_12:0 | 1.33417069 | 1.306083429 | 1.246018195 |
| TG 12:0_12:0_12:1 | 0.312769442 | 0.297152495 | 0.182381794 |
| TG 12:0_12:0_13:0 | 0.329301765 | 0.319973131 | 0.333147907 |
| TG 12:0_12:0_14:0 | 2.670568148 | 2.605408194 | 2.51474175 |
| TG 12:0_12:0_15:0 | 0.476373121 | 0.400537828 | 0.480262538 |
| TG 12:0_12:0_17:0 | 0.423855658 | 0.417172358 | 0.414357689 |
| TG 12:0_12:0_17:1 | 0.256587351 | 0.035868165 | 0.079433725 |
| TG 12:0_12:1_14:0 | 0.911250904 | 0.907551099 | 0.858786024 |
| TG 12:0_12:1_14:3 | 0.002741952 | 0.007897229 | 0.000599403 |
| TG 12:0_12:1_20:5 | 0.001155414 | 0.005413378 | 0.009955272 |
| TG 12:0_12:2_15:0 | 0.036884979 | 0.033884619 | 0.056074986 |
| TG 12:0_12:2_16:0 | 2.084669251 | 2.221848782 | 2.055488503 |
| TG 12:0_12:2_18:2 | 0.01689252 | 0.007657393 | 0.012079444 |
| TG 12:0_14:0_16:0 | 3.518542717 | 3.283197857 | 3.231421904 |
| TG 12:0_14:0_20:4 | 0.012547566 | 0.010680357 | 0.017636791 |
| TG 12:0_15:0_16:0 | 0.383644724 | 0.369414583 | 0.367295525 |
| TG 12:0_15:0_16:0 | 0.120714732 | 0.118094669 | 0.120905955 |
| TG 12:0_16:0_16:0 | 4.049204549 | 3.815514792 | 3.755474309 |
| TG 12:0_16:0_16:0 | 0.001832176 | 0 | 0.00123848 |
| TG 12:0_16:0_18:1 | 3.368177698 | 3.199916559 | 3.166048058 |
| TG 13:0_18:1_18:2 | 0.011325462 | 0.058022314 | 0.012233362 |
| TG 14:0_14:0_16:0 | 0 | 0.004858184 | 0 |
| TG 14:0_14:3_16:0 | 0.002335027 | 0.002701704 | 0.00223507 |
| TG 14:0_14:3_18:0 | 0.000961739 | 0.001664743 | 0.002170224 |
| TG 14:0_15:0_16:0 | 0.468200137 | 0.441498455 | 0.441065967 |
| TG 14:0_15:0_16:0 | 0.177544881 | 0.215428263 | 0.18447307 |
| TG 14:0_15:0_18:1 | 0.542451116 | 0.520667331 | 0.513094973 |
| TG 14:0_16:0_16:0 | 4.927713192 | 4.802666077 | 4.645969341 |
| TG 14:0_16:0_17:1 | 0.039935625 | 0.038427106 | 0.036142854 |
| TG 14:0_16:0_18:0 | 4.702783397 | 5.027203709 | 5.122366878 |
| TG 14:0_16:0_18:1 | 5.243126536 | 5.144237623 | 5.024723319 |
| TG 14:0_16:0_18:1 | 0.005146041 | 0.000700106 | 0.010557488 |
| TG 14:0_16:0_20:4 | 0.032083113 | 0.041413637 | 0.03934032 |
| TG 14:0_16:0_20:5 | 0.006713526 | 0.008268444 | 0.010752006 |
| TG 14:0_18:1_18:2 | 0.885368604 | 0.921190391 | 0.919250885 |
| TG 14:1_16:0_18:1 | 1.856904788 | 1.808117593 | 1.803530634 |
| TG 15:0_16:0_18:0 | 1.107622831 | 1.030161857 | 0.98355993 |
| TG 15:0_16:0_18:1 | 1.103979884 | 1.064487315 | 1.038645807 |
| TG 16:0_16:0_17:1 | 0.072147722 | 0.078385597 | 0.073013093 |
| TG 16:0_16:0_18:0 | 4.223134372 | 4.419063367 | 4.469922927 |
| TG 16:0_16:0_18:1 | 6.318967829 | 7.151852623 | 7.452668293 |
| TG 16:0_16:0_20:4 | 0.05105581 | 0.059963642 | 0.067975775 |
| TG 16:0_16:1_18:1 | 3.419483616 | 3.386333032 | 3.293894393 |
| TG 16:0_17:0_18:0 | 0.673964192 | 0.597613677 | 0.60290225 |
| TG 16:0_17:0_18:1 | 1.202498288 | 1.173595773 | 1.150660013 |
| TG 16:0_17:1_18:0 | 0.059943375 | 0.058316783 | 0.058823723 |
| TG 16:0_18:0_18:0 | 2.412389029 | 2.170649034 | 2.142139779 |
| TG 16:0_18:0_18:1 | 4.971203591 | 5.466532959 | 5.627424868 |
| TG 16:0_18:0_21:0 | 0.06837693 | 0.067588731 | 0.062903836 |
| TG 16:0_18:1_18:1 | 4.949506114 | 5.340677746 | 5.430724443 |
| TG 16:0_18:1_18:2 | 1.733365518 | 1.841590261 | 1.798389909 |
| TG 16:0_18:1_18:3 | 0.368497208 | 0.430714134 | 0.448733832 |
| TG 16:0_18:1_20:5 | 0.015518079 | 0.020093178 | 0.036183 |
| TG 16:0_20:0_22:0 | 0.034853719 | 0.031240561 | 0.026458123 |
| TG 17:0_18:0_18:0 | 0.225595279 | 0.210402363 | 0.19410848 |
| TG 17:0_18:0_18:1 | 0.551210725 | 0.550632443 | 0.549894835 |
| TG 17:0_18:1_18:1 | 0.530718117 | 0.539385164 | 0.578422937 |
| TG 17:1_18:0_18:1 | 0.041902162 | 0.044152897 | 0.036444981 |
| TG 18:0_18:0_18:0 | 0.473121751 | 0.397319818 | 0.386044427 |
| TG 18:0_18:0_18:1 | 1.84101425 | 1.756113699 | 1.778889427 |
| TG 18:0_18:1_18:1 | 2.302990722 | 2.439033233 | 2.479657578 |
| TG 18:1_18:1_18:1 | 2.117680537 | 2.31568233 | 2.308131795 |
| TG 18:1_18:1_18:2 | 0.818718182 | 0.973811903 | 0.919892332 |
| TG 18:1_18:2_18:2 | 0.243702626 | 0.307888565 | 0.316370234 |
| TG 18:1_18:2_18:3 | 0.054860196 | 0.082899693 | 0.090366794 |
| TG 18:1_18:3_18:3 | 0.015267589 | 0.028201654 | 0.024253245 |
| TG 18:1_20:0_22:0 | 0.031709376 | 0.031229894 | 0.031402986 |
| TG 38:2 | 0.108725449 | 0.058747701 | 0.187177827 |
| TG 38:3 | 0.04544691 | 0.026451452 | 0.045675324 |
| TG 40:0 | 2.870659039 | 2.763887264 | 2.65860393 |
| TG 40:1 | 2.398322585 | 2.305424621 | 2.244146286 |
| TG 40:3 | 0.480872884 | 0.56860464 | 0.533636788 |
| TG 40:4 | 0.090699903 | 0.117915076 | 0.038072125 |
| TG 42:1 | 2.446894958 | 2.327003335 | 2.294915533 |
| TG 42:4 | 0.000571206 | 0.003481376 | 0.001456867 |
| TG 43:1 | 0.312286628 | 0.231204359 | 0.292152915 |
| TG 44:1 | 2.802819513 | 2.662554167 | 2.614313933 |
| TG 44:2 | 1.120297139 | 1.098224411 | 1.061818763 |
| TG 44:5 | 0.007426814 | 0.018946165 | 0.016167275 |
| TG 45:1 | 0.354688144 | 0.351208538 | 0.288792189 |
| TG 46:2 | 1.350473716 | 1.265179232 | 1.322314286 |
| TG 46:3 | 0.396845552 | 0.178683059 | 0.411866384 |
| TG 47:0 | 0.979202719 | 0.923655938 | 0.886474424 |
| TG 47:1 | 0.542455836 | 0.520917892 | 0.513064705 |
| TG 47:2 | 0.085921831 | 0.151701258 | 0.137611858 |
| TG 47:3 | 0.007374666 | 0.002980978 | 0.005332527 |
| TG 48:3 | 0.475807326 | 0.101614256 | 0.504344566 |
| TG 48:4 | 0.012662018 | 0.010196948 | 0.023256173 |
| TG 48:5 | 0.006820213 | 0.011931967 | 0.018737204 |
| TG 49:2 | 0.311213266 | 0.297401183 | 0.29508272 |
| TG 51:2 | 0.633500786 | 0.651473936 | 0.625176906 |
| TG 51:4 | 0.013259808 | 0.014503466 | 0.018064455 |
| TG 52:5 | 0.01309407 | 0.003905443 | 0.002693132 |
| TG 52:5 | 0.025707729 | 0.019476045 | 0.018262662 |
| TG 52:6 | 0.002289746 | 0.002816504 | 0.010392927 |
| TG 53:4 | 0.03949827 | 0.008024283 | 0.002306938 |
| TG 54:5 | 0.072666811 | 0.079317816 | 0.079353056 |
| TG 55:1 | 0.157708009 | 0.15707103 | 0.142561128 |
| TG 56:1 | 0.268555333 | 0.257001144 | 0.258640079 |
| TG 56:6 | 0.03357517 | 0.060659362 | 0.026456135 |
| TG 57:0 | 0.031034124 | 0.028323797 | 0.030049486 |
| TG 57:2 | 0.061162947 | 0.0602707 | 0.06027486 |
| TG 58:2 | 0.080067796 | 0.081319971 | 0.07220569 |

TABLE 6C
Raw mass spec data obtained on identified lipid targets obtained
by liquid chromatography with tandem mass spectrometry: LMF-15.

| | LMF-15 | LMF-15 | LMF-15 |
|---|---|---|---|
| Compound Name | Replicate 1 | Replicate 2 | Replicate 3 |
| DG 16:0_16:0 | 0.074675398 | 0.07922151 | 0.075302076 |
| DG 18:0_18:1 | 0.03191818 | 0.033234211 | 0.034417446 |
| PC 18:1_19:1 | 0.0080174 | 0.007644015 | 0.007627554 |
| PC 34:1 | 0.00821994 | 0.009226828 | 0.007905224 |
| PC 36:2 | 0.012280589 | 0.015915574 | 0.013749068 |
| PC 37:2 | 0.005063075 | 0.004306771 | 0.006525816 |
| TG 12:0_12:0_12:0 | 2.2216066 | 2.197261839 | 2.278178719 |
| TG 12:0_12:0_12:1 | 0.563587539 | 0.566356279 | 0.583090841 |
| TG 12:0_12:0_13:0 | 0.54497782 | 0.520716769 | 0.533020605 |
| TG 12:0_12:0_14:0 | 4.075841287 | 3.976198393 | 3.980393432 |
| TG 12:0_12:0_15:0 | 0.356552409 | 0.427514068 | 0.690872957 |
| TG 12:0_12:0_17:0 | 0.346275137 | 0.33429942 | 0.335247146 |
| TG 12:0_12:0_17:1 | 0.50809555 | 0.325866657 | 0.159581232 |
| TG 12:0_12:1_14:0 | 1.588419094 | 1.630846299 | 1.651334745 |
| TG 12:0_12:1_14:3 | 0.004514583 | 0.011225696 | 0.003994158 |
| TG 12:0_12:1_20:5 | 0.007269731 | 0.014904753 | 0.003995342 |
| TG 12:0_12:2_15:0 | 0.021238001 | 0.049851387 | 0.147600652 |
| TG 12:0_12:2_16:0 | 3.844642675 | 3.830155446 | 4.053622896 |
| TG 12:0_12:2_18:2 | 0.016141232 | 0.029607832 | 0.051019113 |
| TG 12:0_14:0_16:0 | 2.561902136 | 2.361372544 | 2.286279123 |
| TG 12:0_14:0_20:4 | 0.029404428 | 0.016898517 | 0.014015289 |
| TG 12:0_15:0_16:0 | 0.209732673 | 0.184729827 | 0.171669971 |
| TG 12:0_15:0_16:0 | 0.118967834 | 0.121863275 | 0.130997759 |
| TG 12:0_16:0_16:0 | 1.573876759 | 1.328297566 | 1.219300168 |
| TG 12:0_16:0_16:0 | 0.002157695 | 0 | 0 |
| TG 12:0_16:0_18:1 | 3.557776769 | 3.463465226 | 3.442622684 |
| TG 13:0_18:1_18:2 | 0.067664798 | 0.073934801 | 0.038258874 |
| TG 14:0_14:0_16:0 | 0.002686039 | 0.002783857 | 0 |
| TG 14:0_14:3_16:0 | 0.004237777 | 0 | 0.003475414 |
| TG 14:0_14:3_18:0 | 0.002123662 | 0.001207409 | 0.002654303 |
| TG 14:0_15:0_16:0 | 0.106009051 | 0.086398468 | 0.074999977 |
| TG 14:0_15:0_16:0 | 0.133236721 | 0.122648917 | 0.128801529 |
| TG 14:0_15:0_18:1 | 0.529013026 | 0.521432643 | 0.471613407 |
| TG 14:0_16:0_16:0 | 0.956418583 | 0.740419691 | 0.685833772 |
| TG 14:0_16:0_17:1 | 0.015666723 | 0.01264211 | 0.013171862 |
| TG 14:0_16:0_18:0 | 0.640647803 | 0.470329392 | 0.437474956 |
| TG 14:0_16:0_18:1 | 4.907613337 | 4.836941944 | 4.822955167 |
| TG 14:0_16:0_18:1 | 0.004514518 | 0.004007577 | 0.005752608 |
| TG 14:0_16:0_20:4 | 0.071447624 | 0.058334882 | 0.0731716 |
| TG 14:0_16:0_20:5 | 0.010006008 | 0.01076081 | 0.010743847 |
| TG 14:0_18:1_18:2 | 1.479820826 | 1.530648655 | 1.53363575 |
| TG 14:1_16:0_18:1 | 2.727553647 | 2.718098289 | 2.72613516 |
| TG 15:0_16:0_18:0 | 0.174437707 | 0.14943788 | 0.134741784 |
| TG 15:0_16:0_18:1 | 0.962778494 | 0.949348517 | 0.93035524 |
| TG 16:0_16:0_17:1 | 0.011120541 | 0.022575792 | 0.018844523 |
| TG 16:0_16:0_18:0 | 0.355966114 | 0.253512841 | 0.220400502 |
| TG 16:0_16:0_18:1 | 6.014358526 | 6.265154099 | 6.042817427 |
| TG 16:0_16:0_20:4 | 0.087517331 | 0.108407512 | 0.093052396 |
| TG 16:0_16:1_18:1 | 4.627522175 | 4.688321366 | 4.715726417 |
| TG 16:0_17:0_18:0 | 0.074071572 | 0.079508092 | 0.061996223 |
| TG 16:0_17:0_18:1 | 0.932486527 | 0.902554275 | 0.908512201 |
| TG 16:0_17:1_18:0 | 0.018851313 | 0.019494957 | 0.021192158 |
| TG 16:0_18:0_18:0 | 0.11895725 | 0.094750166 | 0.096023548 |
| TG 16:0_18:0_18:1 | 4.113344061 | 4.132119807 | 4.000560502 |
| TG 16:0_18:0_21:0 | 0.012045472 | 0.016209989 | 0.01745061 |
| TG 16:0_18:1_18:1 | 6.888264377 | 7.442501349 | 7.360639326 |
| TG 16:0_18:1_18:2 | 3.040718668 | 3.055398657 | 3.129942085 |
| TG 16:0_18:1_18:3 | 0.727009406 | 0.762610454 | 0.769636818 |
| TG 16:0_18:1_20:5 | 0.058456272 | 0.044165372 | 0.05726769 |
| TG 16:0_20:0_22:0 | 0.012941721 | 0.009265732 | 0.003732027 |
| TG 17:0_18:0_18:0 | 0.032425397 | 0.0269943 | 0.02795409 |
| TG 17:0_18:0_18:1 | 0.365719228 | 0.377864745 | 0.37058453 |
| TG 17:0_18:1_18:1 | 0.731939393 | 0.782372112 | 0.774501584 |
| TG 17:1_18:0_18:1 | 0.036092697 | 0.058191197 | 0.057857812 |
| TG 18:0_18:0_18:0 | 0.019071597 | 0.041272316 | 0.047733835 |
| TG 18:0_18:0_18:1 | 0.979973334 | 0.95105258 | 0.93953443 |
| TG 18:0_18:1_18:1 | 3.2358535 | 3.39084842 | 3.391725899 |
| TG 18:1_18:1_18:1 | 3.816954714 | 4.078317571 | 4.12626876 |
| TG 18:1_18:1_18:2 | 1.5982653 | 1.714025231 | 1.735507677 |
| TG 18:1_18:2_18:2 | 0.54481299 | 0.55750938 | 0.561702003 |
| TG 18:1_18:2_18:3 | 0.144727971 | 0.143232607 | 0.160105452 |
| TG 18:1_18:3_18:3 | 0.042263845 | 0.041651721 | 0.045379025 |
| TG 18:1_20:0_22:0 | 0.015690302 | 0.013331848 | 0.0166694 |
| TG 38:2 | 0.737097008 | 0.771857834 | 0.727719856 |
| TG 38:3 | 0.046684284 | 0.01509347 | 0.08515105 |
| TG 40:0 | 3.480822027 | 3.316096674 | 3.23924327 |
| TG 40:1 | 4.007929503 | 4.066200157 | 4.120617847 |
| TG 40:3 | 1.013469639 | 1.034044553 | 1.052031232 |
| TG 40:4 | 0.199384959 | 0.219567412 | 0.180358909 |
| TG 42:1 | 3.755297497 | 3.705863509 | 3.711244376 |
| TG 42:4 | 0.04741849 | 0.048175758 | 0.005604033 |
| TG 43:1 | 0.434393355 | 0.362236021 | 0.363999809 |
| TG 44:1 | 3.58543397 | 3.498326356 | 3.570622928 |
| TG 44:2 | 1.916025705 | 1.885409485 | 1.951737988 |
| TG 44:5 | 0.036105552 | 0.025123214 | 0.027577723 |
| TG 45:1 | 0.424261482 | 0.434300065 | 0.441540744 |
| TG 46:2 | 2.129073036 | 2.142250451 | 2.198801709 |
| TG 46:3 | 0.731867937 | 0.738169013 | 0.72360007 |
| TG 47:0 | 0.223326688 | 0.204729837 | 0.173695339 |
| TG 47:1 | 0.529454737 | 0.521432643 | 0.53762306 |
| TG 47:2 | 0.183384513 | 0.045174321 | 0.226137791 |
| TG 47:3 | 0.018250663 | 0.03556461 | 0.021338069 |
| TG 48:3 | 0.819667354 | 0.888021422 | 0.865493908 |
| TG 48:4 | 0.035741503 | 0.04065518 | 0.037729155 |
| TG 48:5 | 0.022506586 | 0.030236397 | 0.020108608 |
| TG 49:2 | 0.379299395 | 0.441194358 | 0.398921122 |
| TG 51:2 | 0.847290608 | 0.8958577 | 0.884816236 |
| TG 51:4 | 0.007833551 | 0.044850753 | 0.028845182 |
| TG 52:5 | 0.021848511 | 0.024440459 | 0.009100726 |
| TG 52:5 | 0.116979676 | 0.084597009 | 0.019270755 |
| TG 52:6 | 0 | 0 | 0.016028531 |
| TG 53:4 | 0.012254491 | 0.011874362 | 0.055852225 |
| TG 54:5 | 0.126277231 | 0.152031521 | 0.15821621 |
| TG 55:1 | 0.076002384 | 0.063032108 | 0.076231533 |
| TG 56:1 | 0.094791592 | 0.097402135 | 0.092501337 |
| TG 56:6 | 0.050823156 | 0.104656478 | 0.061082813 |
| TG 57:0 | 0.012941371 | 0.015509625 | 0.013036341 |
| TG 57:2 | 0.053459001 | 0.054617458 | 0.067106733 |
| TG 58:2 | 0.076150068 | 0.077836619 | 0.087554572 |

TABLE 6D
Raw mass spec data obtained on identified lipid targets obtained
by liquid chromatography with tandem mass spectrometry: HMF-25.

| | HMF-25 | HMF-25 | HMF-25 |
|---|---|---|---|
| Compound Name | Replicate 1 | Replicate 2 | Replicate 3 |
| DG 16:0_16:0 | 0.087727818 | 0.104432559 | 0.066839214 |
| DG 18:0_18:1 | 0.035127201 | 0 | 0.024467584 |
| PC 18:1_19:1 | 0.032489836 | 0.037240708 | 0.008945855 |
| PC 34:1 | 0.027652112 | 0.034427593 | 0.010174949 |
| PC 36:2 | 0.056013603 | 0.057424478 | 0.014987827 |
| PC 37:2 | 0.024357393 | 0.025373629 | 0.006249598 |
| TG 12:0_12:0_12:0 | 1.457260816 | 1.613623329 | 1.415710284 |
| TG 12:0_12:0_12:1 | 0.184199755 | 0.315033234 | 0.379584003 |
| TG 12:0_12:0_13:0 | 0.365856022 | 0.419025551 | 0.332139899 |
| TG 12:0_12:0_14:0 | 2.666889137 | 0.658536647 | 0.583708689 |
| TG 12:0_12:0_15:0 | 0.258681103 | 0.283540939 | 0.487672261 |
| TG 12:0_12:0_17:0 | 0.346195953 | 0.400050044 | 0.3985408 |
| TG 12:0_12:0_17:1 | 0.294251897 | 0.338755443 | 0.298535259 |
| TG 12:0_12:1_14:0 | 0.974067963 | 1.11016049 | 1.011936717 |
| TG 12:0_12:1_14:3 | 0.007038194 | 0.005957586 | 0.001400746 |
| TG 12:0_12:1_20:5 | 0.016856589 | 0.022596532 | 0.003928555 |
| TG 12:0_12:2_15:0 | 0.097107687 | 0.048994478 | 0.021169274 |
| TG 12:0_12:2_16:0 | 2.25158608 | 2.521351484 | 2.470207862 |
| TG 12:0_12:2_18:2 | 0.007351485 | 0.00980989 | 0.012206841 |
| TG 12:0_14:0_16:0 | 2.878166195 | 2.758210105 | 2.995731598 |
| TG 12:0_14:0_20:4 | 0.012157854 | 0.008250944 | 0.018421401 |
| TG 12:0_15:0_16:0 | 0.364875689 | 0.351516518 | 0.345243907 |
| TG 12:0_15:0_16:0 | 0.120282346 | 0.139486906 | 0.101967859 |
| TG 12:0_16:0_16:0 | 3.109853811 | 2.880967909 | 3.445502269 |
| TG 12:0_16:0_16:0 | 0.003448947 | 0 | 0.001272034 |
| TG 12:0_16:0_18:1 | 2.966038307 | 3.019331815 | 3.198704563 |
| TG 13:0_18:1_18:2 | 0.025826516 | 0.038050163 | 0.017336499 |
| TG 14:0_14:0_16:0 | 0 | 0 | 0 |
| TG 14:0_14:3_16:0 | 0.001803052 | 0.009445463 | 0.002916457 |
| TG 14:0_14:3_18:0 | 0.001702331 | 0 | 0.003482451 |
| TG 14:0_15:0_16:0 | 0.440169334 | 0.3908777 | 0.396860609 |
| TG 14:0_15:0_16:0 | 0.192415978 | 0.163255408 | 0.156874329 |
| TG 14:0_15:0_18:1 | 0.434823546 | 0.516111917 | 0.521297039 |
| TG 14:0_16:0_16:0 | 3.733514982 | 3.421197627 | 4.377989225 |
| TG 14:0_16:0_17:1 | 0.024428547 | 0.029472633 | 0.026448939 |
| TG 14:0_16:0_18:0 | 4.792858952 | 4.335650299 | 4.883374486 |
| TG 14:0_16:0_18:1 | 4.559370872 | 4.541002348 | 5.089030968 |
| TG 14:0_16:0_18:1 | 0.041140564 | 0 | 0.005818421 |
| TG 14:0_16:0_20:4 | 0.03577889 | 0.033197284 | 0.0468509 |
| TG 14:0_16:0_20:5 | 0.006199487 | 0.018053109 | 0.008216891 |
| TG 14:0_18:1_18:2 | 0.951583131 | 1.035695462 | 1.024287833 |
| TG 14:1_16:0_18:1 | 1.865051056 | 1.988902518 | 1.952331716 |
| TG 15:0_16:0_18:0 | 0.839849585 | 0.844191494 | 0.939296916 |
| TG 15:0_16:0_18:1 | 1.06063105 | 0.992852346 | 1.016588094 |
| TG 16:0_16:0_17:1 | 0.056404157 | 0.056670784 | 0.067595166 |
| TG 16:0_16:0_18:0 | 4.341812078 | 4.01905728 | 4.344466879 |
| TG 16:0_16:0_18:1 | 8.056803396 | 7.966426397 | 7.285140067 |
| TG 16:0_16:0_20:4 | 0.040055396 | 0.061454989 | 0.063287522 |
| TG 16:0_16:1_18:1 | 3.30165775 | 3.457107735 | 3.524050758 |
| TG 16:0_17:0_18:0 | 0.537923747 | 0.511729878 | 0.581889576 |
| TG 16:0_17:0_18:1 | 1.128993248 | 1.146362602 | 1.124605525 |
| TG 16:0_17:1_18:0 | 0.076744905 | 0.080265914 | 0.049335323 |
| TG 16:0_18:0_18:0 | 1.956195409 | 1.880356516 | 2.151500539 |
| TG 16:0_18:0_18:1 | 5.921143989 | 5.80223199 | 5.445275758 |
| TG 16:0_18:0_21:0 | 0.056996544 | 0.070619162 | 0.058138408 |
| TG 16:0_18:1_18:1 | 5.837965358 | 6.161905048 | 5.780668993 |
| TG 16:0_18:1_18:2 | 1.895223022 | 2.092141686 | 2.021797033 |
| TG 16:0_18:1_18:3 | 0.467247813 | 0.55134271 | 0.496194022 |
| TG 16:0_18:1_20:5 | 0.058786961 | 0.056992424 | 0.035336521 |
| TG 16:0_20:0_22:0 | 0.045981497 | 0.037049257 | 0.027821051 |
| TG 17:0_18:0_18:0 | 0.212494652 | 0.18640198 | 0.200169168 |
| TG 17:0_18:0_18:1 | 0.589737673 | 0.58892708 | 0.502851095 |
| TG 17:0_18:1_18:1 | 0.633025992 | 0.664864915 | 0.594028307 |
| TG 17:1_18:0_18:1 | 0.037124933 | 0.044603662 | 0.046345841 |
| TG 18:0_18:0_18:0 | 0.402276088 | 0.440653021 | 0.405045525 |
| TG 18:0_18:0_18:1 | 1.814694521 | 1.803086323 | 1.729685255 |
| TG 18:0_18:1_18:1 | 2.762592567 | 2.976312965 | 2.597483391 |
| TG 18:1_18:1_18:1 | 2.662306792 | 2.914281199 | 2.608223462 |
| TG 18:1_18:1_18:2 | 1.038808351 | 1.117198885 | 1.067371586 |
| TG 18:1_18:2_18:2 | 0.284033409 | 0.365870836 | 0.322598635 |
| TG 18:1_18:2_18:3 | 0.077872361 | 0.088767523 | 0.068946668 |
| TG 18:1_18:3_18:3 | 0.036862212 | 0.042288624 | 0.032305014 |
| TG 18:1_20:0_22:0 | 0.045668043 | 0.050158099 | 0.030809044 |
| TG 38:2 | 0.28682814 | 0.005186608 | 0.061989455 |
| TG 38:3 | 0.01360915 | 0.005486962 | 0.011384744 |
| TG 40:0 | 2.668473619 | 2.782392366 | 2.562027024 |
| TG 40:1 | 2.388034965 | 2.631254546 | 2.535127681 |
| TG 40:3 | 0.58319691 | 0.690850112 | 0.641054425 |
| TG 40:4 | 0.131067329 | 0.033915579 | 0.127248442 |
| TG 42:1 | 2.361551381 | 2.617889762 | 2.44583078 |
| TG 42:4 | 0.008395798 | 0.008816469 | 0.005285355 |
| TG 43:1 | 0.296677226 | 0.299116341 | 0.296978304 |
| TG 44:1 | 2.583291792 | 2.655469396 | 2.718090486 |
| TG 44:2 | 1.03877974 | 1.170330889 | 1.194301319 |
| TG 44:5 | 0.013310868 | 0.027354828 | 0.017979466 |
| TG 45:1 | 0.369401165 | 0.336722902 | 0.293136724 |
| TG 46:2 | 1.343895493 | 1.448408838 | 1.442621271 |
| TG 46:3 | 0.121165654 | 0.462755694 | 0.451592337 |
| TG 47:0 | 0.82306192 | 0.757783218 | 0.826081699 |
| TG 47:1 | 0.434823546 | 0.500135799 | 0.521297039 |
| TG 47:2 | 0.117183008 | 0.178191806 | 0.141244892 |
| TG 47:3 | 0.017664253 | 0.022675384 | 0.007209064 |
| TG 48:3 | 0.492901954 | 0.526914918 | 0.553869353 |
| TG 48:4 | 0.019808914 | 0.027515184 | 0.021453207 |
| TG 48:5 | 0.027509725 | 0.012481726 | 0.014473965 |
| TG 49:2 | 0.28285282 | 0.322120078 | 0.323962452 |
| TG 51:2 | 0.590752329 | 0.752078275 | 0.665237192 |
| TG 51:4 | 0.02577173 | 0.037571919 | 0.017824198 |
| TG 52:5 | 0.024328682 | 0.023363647 | 0.006426323 |
| TG 52:5 | 0.076919154 | 0.054522596 | 0.049390615 |
| TG 52:6 | 0.002414795 | 0.002978884 | 0.000722302 |
| TG 53:4 | 0.013946097 | 0.030430875 | 0.021857609 |
| TG 54:5 | 0.088195755 | 0.105718325 | 0.054739242 |
| TG 55:1 | 0.195656348 | 0.209161452 | 0.153565835 |
| TG 56:1 | 0.291502206 | 0.309405014 | 0.242264511 |
| TG 56:6 | 0.051853913 | 0.050693531 | 0.034158989 |
| TG 57:0 | 0.047832286 | 0.057687992 | 0.028687215 |
| TG 57:2 | 0.053563658 | 0.009190336 | 0.050084634 |
| TG 58:2 | 0.083663145 | 0.07624368 | 0.073584094 |

TABLE 6E
Raw mass spec data obtained on identified lipid targets obtained
by liquid chromatography with tandem mass spectrometry: LMF-25.

| | LMF-25 | LMF-25 | LMF-25 |
|---|---|---|---|
| Compound Name | Replicate 1 | Replicate 2 | Replicate 3 |
| DG 16:0_16:0 | 0.08292381 | 0.08364686 | 0.0751917 |
| DG 18:0_18:1 | 0.034430447 | 0.017755208 | 0.033088665 |
| PC 18:1_19:1 | 0.011345862 | 0.009925091 | 0.007841279 |
| PC 34:1 | 0.010614635 | 0.008985988 | 0.007603842 |
| PC 36:2 | 0.015200947 | 0.015045792 | 0.01191412 |
| PC 37:2 | 0.006783427 | 0.007677061 | 0.005612863 |
| TG 12:0_12:0_12:0 | 2.204436357 | 2.173639384 | 2.21203347 |
| TG 12:0_12:0_12:1 | 0.462821029 | 0.549883604 | 0.550475219 |
| TG 12:0_12:0_13:0 | 0.503819582 | 0.527873295 | 0.516117856 |
| TG 12:0_12:0_14:0 | 4.038189099 | 4.048530609 | 4.200425428 |
| TG 12:0_12:0_15:0 | 0.364930937 | 0.71662007 | 0.650737351 |
| TG 12:0_12:0_17:0 | 0.361660898 | 0.360714423 | 0.362930604 |
| TG 12:0_12:0_17:1 | 0.499270195 | 0.525010088 | 0.145537251 |
| TG 12:0_12:1_14:0 | 1.58576213 | 1.593405814 | 1.61338973 |
| TG 12:0_12:1_14:3 | 0.003797322 | 0.003767055 | 0.013693908 |
| TG 12:0_12:1_20:5 | 0.015994904 | 0.014516028 | 0.002678762 |
| TG 12:0_12:2_15:0 | 0.068666297 | 0.108983066 | 0.040743674 |
| TG 12:0_12:2_16:0 | 3.818240598 | 3.855492461 | 3.793736786 |
| TG 12:0_12:2_18:2 | 0.00365199 | 0.024494362 | 0.083117011 |
| TG 12:0_14:0_16:0 | 2.584092794 | 2.562422912 | 2.668976522 |
| TG 12:0_14:0_20:4 | 0.015014347 | 0.028003274 | 0.03027237 |
| TG 12:0_15:0_16:0 | 0.206418756 | 0.164839118 | 0.200418939 |
| TG 12:0_15:0_16:0 | 0.132524672 | 0.12635806 | 0.130342989 |
| TG 12:0_16:0_16:0 | 1.51757335 | 1.391180864 | 1.546099825 |
| TG 12:0_16:0_16:0 | 0.001980627 | 0.001444779 | 0 |
| TG 12:0_16:0_18:1 | 3.499436892 | 3.332064637 | 3.648631345 |
| TG 13:0_18:1_18:2 | 0.065710379 | 0.062557989 | 0.016311654 |
| TG 14:0_14:0_16:0 | 0 | 0 | 0 |
| TG 14:0_14:3_16:0 | 0.001053505 | 0.002286334 | 0 |
| TG 14:0_14:3_18:0 | 0.002636853 | 0.005098055 | 0.002848979 |
| TG 14:0_15:0_16:0 | 0.099124195 | 0.08967871 | 0.087536323 |
| TG 14:0_15:0_16:0 | 0.147882624 | 0.138342979 | 0.150398661 |
| TG 14:0_15:0_18:1 | 0.545975365 | 0.512814946 | 0.538501046 |
| TG 14:0_16:0_16:0 | 0.855955626 | 0.791250273 | 0.847856191 |
| TG 14:0_16:0_17:1 | 0.015678953 | 0.014926271 | 0.015420499 |
| TG 14:0_16:0_18:0 | 0.552027367 | 0.505546546 | 0.560937015 |
| TG 14:0_16:0_18:1 | 4.975120485 | 4.841218014 | 4.905665897 |
| TG 14:0_16:0_18:1 | 0.010650424 | 0.008428861 | 0.005525004 |
| TG 14:0_16:0_20:4 | 0.039232711 | 0.068130734 | 0.068383492 |
| TG 14:0_16:0_20:5 | 0.020809154 | 0.015091999 | 0.013062347 |
| TG 14:0_18:1_18:2 | 1.45846712 | 1.424506602 | 1.485917391 |
| TG 14:1_16:0_18:1 | 2.656361373 | 2.587040123 | 2.740150717 |
| TG 15:0_16:0_18:0 | 0.165790647 | 0.149636711 | 0.152319438 |
| TG 15:0_16:0_18:1 | 0.972379748 | 0.966044089 | 0.953064143 |
| TG 16:0_16:0_17:1 | 0.024384227 | 0.024189454 | 0.02065462 |
| TG 16:0_16:0_18:0 | 0.288031214 | 0.248848739 | 0.312061514 |
| TG 16:0_16:0_18:1 | 6.540049801 | 6.470812704 | 5.927080071 |
| TG 16:0_16:0_20:4 | 0.080476933 | 0.087738497 | 0.096540132 |
| TG 16:0_16:1_18:1 | 4.649822193 | 4.580333075 | 4.614018464 |
| TG 16:0_17:0_18:0 | 0.062556639 | 0.060497149 | 0.089995876 |
| TG 16:0_17:0_18:1 | 0.955504895 | 0.914933075 | 0.944163958 |
| TG 16:0_17:1_18:0 | 0.022218583 | 0.021837776 | 0.020540039 |
| TG 16:0_18:0_18:0 | 0.104139289 | 0.102212484 | 0.119031911 |
| TG 16:0_18:0_18:1 | 4.418054282 | 4.299510899 | 4.134858421 |
| TG 16:0_18:0_21:0 | 0.021864064 | 0.014227246 | 0.019139847 |
| TG 16:0_18:1_18:1 | 7.446816485 | 7.423683112 | 6.784393004 |
| TG 16:0_18:1_18:2 | 2.964566518 | 2.939399194 | 3.001155877 |
| TG 16:0_18:1_18:3 | 0.716688127 | 0.713557003 | 0.725143257 |
| TG 16:0_18:1_20:5 | 0.025905709 | 0.052331145 | 0.043053941 |
| TG 16:0_20:0_22:0 | 0.014197831 | 0.007708108 | 0.001945485 |
| TG 17:0_18:0_18:0 | 0.028239511 | 0.026872132 | 0.026309833 |
| TG 17:0_18:0_18:1 | 0.318181584 | 0.360435198 | 0.372261311 |
| TG 17:0_18:1_18:1 | 0.743220838 | 0.766158692 | 0.731289523 |
| TG 17:1_18:0_18:1 | 0.040013169 | 0.050653774 | 0.036069921 |
| TG 18:0_18:0_18:0 | 0.047051312 | 0.051836583 | 0.045922254 |
| TG 18:0_18:0_18:1 | 0.994373485 | 0.996120785 | 1.035749422 |
| TG 18:0_18:1_18:1 | 3.387460636 | 3.331995486 | 3.198478477 |
| TG 18:1_18:1_18:1 | 3.975576655 | 3.986027872 | 3.736814711 |
| TG 18:1_18:1_18:2 | 1.677926388 | 1.584350466 | 1.576250004 |
| TG 18:1_18:2_18:2 | 0.536597144 | 0.516698082 | 0.539900348 |
| TG 18:1_18:2_18:3 | 0.113369187 | 0.108998421 | 0.137407565 |
| TG 18:1_18:3_18:3 | 0.045556605 | 0.047348512 | 0.044322988 |
| TG 18:1_20:0_22:0 | 0.020518142 | 0.017573543 | 0.018962337 |
| TG 38:2 | 0.135317136 | 0.402337397 | 0.749729188 |
| TG 38:3 | 0.030501698 | 0.083874232 | 0.015902545 |
| TG 40:0 | 3.440609944 | 3.489666749 | 3.564404095 |
| TG 40:1 | 3.876127764 | 4.04882468 | 4.048566918 |
| TG 40:3 | 1.012744283 | 0.928938693 | 0.986743678 |
| TG 40:4 | 0.087383672 | 0.206647031 | 0.120213784 |
| TG 42:1 | 3.653080013 | 3.722658751 | 3.752693846 |
| TG 42:4 | 0.000712981 | 0.014297507 | 0.046091215 |
| TG 43:1 | 0.437735343 | 0.453045843 | 0.437347052 |
| TG 44:1 | 3.513861119 | 3.523726657 | 3.62626776 |
| TG 44:2 | 1.829455372 | 1.899155117 | 1.88264567 |
| TG 44:5 | 0.032072401 | 0.018458276 | 0.030919598 |
| TG 45:1 | 0.444729224 | 0.399964207 | 0.437003944 |
| TG 46:2 | 2.104339946 | 2.128012696 | 2.136142458 |
| TG 46:3 | 0.719193935 | 0.723226258 | 0.710777845 |
| TG 47:0 | 0.212444508 | 0.198379123 | 0.213195006 |
| TG 47:1 | 0.543858824 | 0.512814946 | 0.538071008 |
| TG 47:2 | 0.238823526 | 0.230185989 | 0.246049969 |
| TG 47:3 | 0 | 0.047866822 | 0.040388519 |
| TG 48:3 | 0.819711353 | 0.857471904 | 0.851110947 |
| TG 48:4 | 0.032737985 | 0.031313247 | 0.022081598 |
| TG 48:5 | 0.020560995 | 0.01823703 | 0.016557939 |
| TG 49:2 | 0.423314674 | 0.332530743 | 0.437366438 |
| TG 51:2 | 0.887130339 | 0.887080189 | 0.863854711 |
| TG 51:4 | 0.010127386 | 0.018543484 | 0.048334575 |
| TG 52:5 | 0.010057832 | 0.005448588 | 0.028206814 |
| TG 52:5 | 0.02099002 | 0.005448872 | 0.085928046 |
| TG 52:6 | 0.00251482 | 0.000600462 | 0.001057459 |
| TG 53:4 | 0.038545257 | 0.043945246 | 0.05994749 |
| TG 54:5 | 0.151068037 | 0.125866931 | 0.144552072 |
| TG 55:1 | 0.06813333 | 0.062495827 | 0.071452062 |
| TG 56:1 | 0.091173559 | 0.087162409 | 0.082773554 |
| TG 56:6 | 0.037285173 | 0.072356834 | 0.10026958 |
| TG 57:0 | 0.014875853 | 0.0118588 | 0.0095572 |
| TG 57:2 | 0.06837249 | 0.074702117 | 0.055553354 |
| TG 58:2 | 0.092609326 | 0.091087994 | 0.089216641 |

TABLE 6F
Raw mass spec data obtained on identified lipid targets obtained
by liquid chromatography with tandem mass spectrometry: AMF.

| | AMF | AMF | AMF |
|---|---|---|---|
| Compound Name | Replicate 1 | Replicate 2 | Replicate 3 |
| DG 16:0_16:0 | 0.07700043 | 0.087151802 | 0.06501985 |
| DG 18:0_18:1 | 0.031994028 | 0.027749506 | 0.02659497 |
| PC 18:1_19:1 | 0.013072543 | 0.020343013 | 0.006917932 |
| PC 34:1 | 0.011844406 | 0.023632621 | 0.006977138 |
| PC 36:2 | 0.022141559 | 0.034193101 | 0.011122267 |
| PC 37:2 | 0.009204478 | 0.014122682 | 0.004218691 |
| TG 12:0_12:0_12:0 | 1.703884233 | 1.744121638 | 1.783664753 |
| TG 12:0_12:0_12:1 | 0.448657482 | 0.28524621 | 0.454448714 |
| TG 12:0_12:0_13:0 | 0.443449086 | 0.442614242 | 0.42700045 |
| TG 12:0_12:0_14:0 | 0.704973301 | 3.17210206 | 3.559465903 |
| TG 12:0_12:0_15:0 | 0.570008313 | 0.574220671 | 0.59066953 |
| TG 12:0_12:0_17:0 | 0.3339353 | 0.426999274 | 0.43469695 |
| TG 12:0_12:0_17:1 | 0.380925289 | 0.34316863 | 0.395406841 |
| TG 12:0_12:1_14:0 | 1.221198428 | 1.197532099 | 1.270279634 |
| TG 12:0_12:1_14:3 | 0.007453902 | 0.003912377 | 0.003153121 |
| TG 12:0_12:1_20:5 | 0.000930108 | 0.010678576 | 0.008039605 |
| TG 12:0_12:2_15:0 | 0.024567781 | 0.041508742 | 0.083895992 |
| TG 12:0_12:2_16:0 | 3.065281404 | 3.079603275 | 3.042157607 |
| TG 12:0_12:2_18:2 | 0.006071273 | 0.055109594 | 0.03329877 |
| TG 12:0_14:0_16:0 | 2.683991887 | 2.591169703 | 2.862935544 |
| TG 12:0_14:0_20:4 | 0.01554744 | 0.007729466 | 0.011866544 |
| TG 12:0_15:0_16:0 | 0.277055897 | 0.272358445 | 0.267079583 |
| TG 12:0_15:0_16:0 | 0.109248198 | 0.120132205 | 0.111688604 |
| TG 12:0_16:0_16:0 | 2.433572383 | 2.282961847 | 2.633239659 |
| TG 12:0_16:0_16:0 | 0.002016797 | 0.002000073 | 0.005528301 |
| TG 12:0_16:0_18:1 | 3.191621084 | 3.052874989 | 3.296613893 |
| TG 13:0_18:1_18:2 | 0.031776931 | 0.01770822 | 0.023224005 |
| TG 14:0_14:0_16:0 | 0 | 0 | 0.002801452 |
| TG 14:0_14:3_16:0 | 0.002433665 | 0.005655754 | 0.001105378 |
| TG 14:0_14:3_18:0 | 0.001806048 | 0 | 0.002169407 |
| TG 14:0_15:0_16:0 | 0.26822491 | 0.273769493 | 0.262658333 |
| TG 14:0_15:0_16:0 | 0.106196524 | 0.146472158 | 0.145207738 |
| TG 14:0_15:0_18:1 | 0.53245595 | 0.541786064 | 0.51290625 |
| TG 14:0_16:0_16:0 | 2.758783588 | 2.458971759 | 2.945778785 |
| TG 14:0_16:0_17:1 | 0.026899227 | 0.021649458 | 0.024416837 |
| TG 14:0_16:0_18:0 | 3.274469524 | 3.031654388 | 3.022270685 |
| TG 14:0_16:0_18:1 | 4.938295972 | 4.476280912 | 4.930448928 |
| TG 14:0_16:0_18:1 | 0.010324584 | 0.002293542 | 0.001384243 |
| TG 14:0_16:0_20:4 | 0.057424412 | 0.065669376 | 0.043679979 |
| TG 14:0_16:0_20:5 | 0.001783588 | 0.020339288 | 0.012994572 |
| TG 14:0_18:1_18:2 | 1.206299908 | 1.216885863 | 1.154109325 |
| TG 14:1_16:0_18:1 | 2.257750535 | 2.225973697 | 2.196140094 |
| TG 15:0_16:0_18:0 | 0.608328494 | 0.575336216 | 0.614174128 |
| TG 15:0_16:0_18:1 | 1.002443739 | 0.915747956 | 1.021576796 |
| TG 16:0_16:0_17:1 | 0.049081236 | 0.038646246 | 0.051256663 |
| TG 16:0_16:0_18:0 | 2.936599912 | 2.686197192 | 2.796421572 |
| TG 16:0_16:0_18:1 | 7.550734206 | 7.560841924 | 5.966090395 |
| TG 16:0_16:0_20:4 | 0.066566817 | 0.075841349 | 0.072694702 |
| TG 16:0_16:1_18:1 | 3.914625885 | 3.753856543 | 3.997968681 |
| TG 16:0_17:0_18:0 | 0.384161354 | 0.368277725 | 0.384683056 |
| TG 16:0_17:0_18:1 | 1.048111711 | 0.992622169 | 1.032309876 |
| TG 16:0_17:1_18:0 | 0.03803704 | 0.028827318 | 0.032880481 |
| TG 16:0_18:0_18:0 | 1.372353411 | 1.247117451 | 1.448368673 |
| TG 16:0_18:0_18:1 | 5.33334875 | 5.255992985 | 4.500238994 |
| TG 16:0_18:0_21:0 | 0.051731053 | 0.059653532 | 0.03891537 |
| TG 16:0_18:1_18:1 | 6.96284474 | 6.959175354 | 5.731748617 |
| TG 16:0_18:1_18:2 | 2.45546346 | 2.412293796 | 2.454741141 |
| TG 16:0_18:1_18:3 | 0.610782645 | 0.607411885 | 0.559319191 |
| TG 16:0_18:1_20:5 | 0.022431136 | 0.055252565 | 0.036090463 |
| TG 16:0_20:0_22:0 | 0.024689791 | 0.040635883 | 0.022604719 |
| TG 17:0_18:0_18:0 | 0.110378793 | 0.144445276 | 0.128391169 |
| TG 17:0_18:0_18:1 | 0.448883568 | 0.395344778 | 0.472701028 |
| TG 17:0_18:1_18:1 | 0.693920932 | 0.694691255 | 0.626585189 |
| TG 17:1_18:0_18:1 | 0.044505135 | 0.03283075 | 0.037680749 |
| TG 18:0_18:0_18:0 | 0.273416243 | 0.286366982 | 0.273976153 |
| TG 18:0_18:0_18:1 | 1.467910039 | 1.466851314 | 1.448330034 |
| TG 18:0_18:1_18:1 | 3.073262941 | 3.019481761 | 2.712504612 |
| TG 18:1_18:1_18:1 | 3.346366762 | 3.274684792 | 2.937591013 |
| TG 18:1_18:1_18:2 | 1.334641317 | 1.325197216 | 1.258529094 |
| TG 18:1_18:2_18:2 | 0.362267619 | 0.406723852 | 0.356565368 |
| TG 18:1_18:2_18:3 | 0.12488557 | 0.140051459 | 0.104246905 |
| TG 18:1_18:3_18:3 | 0.038921455 | 0.05485791 | 0.033849949 |
| TG 18:1_20:0_22:0 | 0.015741103 | 0.024492197 | 0.024108278 |
| TG 38:2 | 0.189922048 | 0.114382987 | 0.07921702 |
| TG 38:3 | 0.030944225 | 0.007739121 | 0.018912327 |
| TG 40:0 | 2.832381655 | 2.882605922 | 3.218468364 |
| TG 40:1 | 3.018264302 | 3.066146053 | 3.239348387 |
| TG 40:3 | 0.80460911 | 0.730168482 | 0.829403125 |
| TG 40:4 | 0.139885756 | 0.068867867 | 0.151157017 |
| TG 42:1 | 2.868275788 | 2.910571674 | 3.172164383 |
| TG 42:4 | 0.016123624 | 0 | 0.03361355 |
| TG 43:1 | 0.363557285 | 0.343526966 | 0.372582617 |
| TG 44:1 | 2.92915913 | 2.913836006 | 3.043457768 |
| TG 44:2 | 1.430989032 | 1.413936857 | 1.530657236 |
| TG 44:5 | 0.030116748 | 0.012400428 | 0.01870016 |
| TG 45:1 | 0.389288781 | 0.36664725 | 0.375458092 |
| TG 46:2 | 1.700217844 | 1.724878311 | 1.674131546 |
| TG 46:3 | 0.533013454 | 0.507475762 | 0.589671534 |
| TG 47:0 | 0.565650696 | 0.555891669 | 0.581152684 |
| TG 47:1 | 0.53253289 | 0.54442454 | 0.51373563 |
| TG 47:2 | 0.082983299 | 0.141936854 | 0.162318417 |
| TG 47:3 | 0.034298012 | 0.018948184 | 0.020749858 |
| TG 48:3 | 0.653480974 | 0.485503415 | 0.65829987 |
| TG 48:4 | 0.027266051 | 0.019077109 | 0.031409068 |
| TG 48:5 | 0.023400908 | 0.0186315 | 0.016401492 |
| TG 49:2 | 0.368531648 | 0.240981708 | 0.353462816 |
| TG 51:2 | 0.752772534 | 0.761634904 | 0.749308587 |
| TG 51:4 | 0.009869616 | 0.023202892 | 0.011268376 |
| TG 52:5 | 0.003073694 | 0.015865027 | 0.024267221 |
| TG 52:5 | 0.039657437 | 0.075739577 | 0.092455793 |
| TG 52:6 | 0.003961679 | 0.001022241 | 0.009053573 |
| TG 53:4 | 0.006860987 | 0.006853525 | 0.036227879 |
| TG 54:5 | 0.055759621 | 0.067596308 | 0.040495262 |
| TG 55:1 | 0.081275717 | 0.1495564 | 0.125534446 |
| TG 56:1 | 0.224792689 | 0.21970348 | 0.187184989 |
| TG 56:6 | 0.056141959 | 0.04847447 | 0.029618946 |
| TG 57:0 | 0.02714897 | 0.045656037 | 0.020491599 |
| TG 57:2 | 0.065521307 | 0.081586644 | 0.054682896 |
| TG 58:2 | 0.076261277 | 0.096435389 | 0.080517554 |

### Milkfat-Based Intravenous Fat Emulsion (IVFE) Preparation

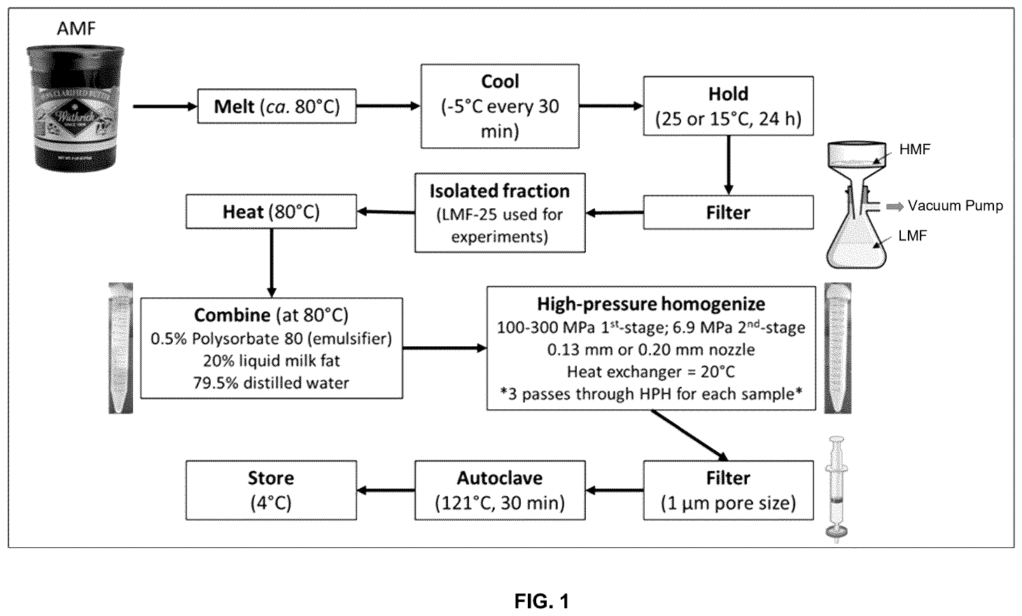

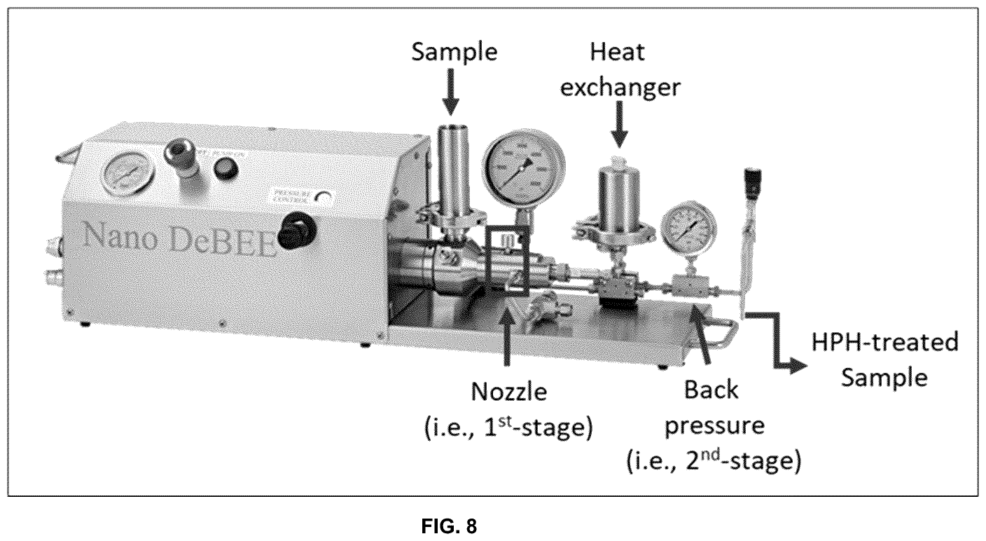

Distilled water with 0.50% (w/w) polysorbate 80 was heated to 80° C. Selected AMF fraction(s) (80° C.) were added to the solution at 20% (w/w) under constant agitation. Samples were then processed using ultra high-pressure homogenization (UHPH, Nano DeBEE 45-2, BEE International, South Easton, MA, FIG. 8). Samples were UHPH-treated with three passes at 100, 200, or 300 MPa first-stage pressure and 6.9 MPa second-stage pressure (Table 7). Samples were immediately cooled in the in-line heat exchanger with a co-current flow of water (20° C.). After processing, samples were filtered (1.0 μm pore size, Catalog #76479-036, VWR International, 10 Radnor, PA) and autoclaved (121° C., 30 min) to ensure adequate particle size and sterility prior to analysis and intravenous application. The entire process is depicted in the schematic of FIG. 1.

TABLE 7
Ultra high-pressure homogenization conditions

| First-stage | Second-stage | Orifice | # of | Heat exchanger |
|---|---|---|---|---|
| Pressure (MPa) | Pressure (MPa) | size (mm) | passes | temperature (° C.) |
| 100 | 6.9 | 0.20 | 3 | 20 |
| 200 | 6.9 | 0.13 | 3 | 20 |
| 300 | 6.9 | 0.13 | 3 | 20 |

### Nanoparticle Size Stability

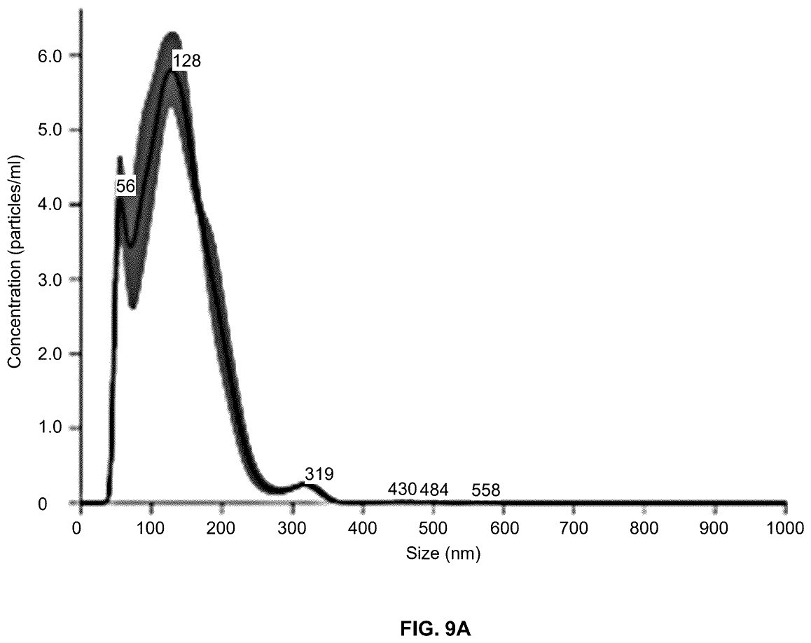

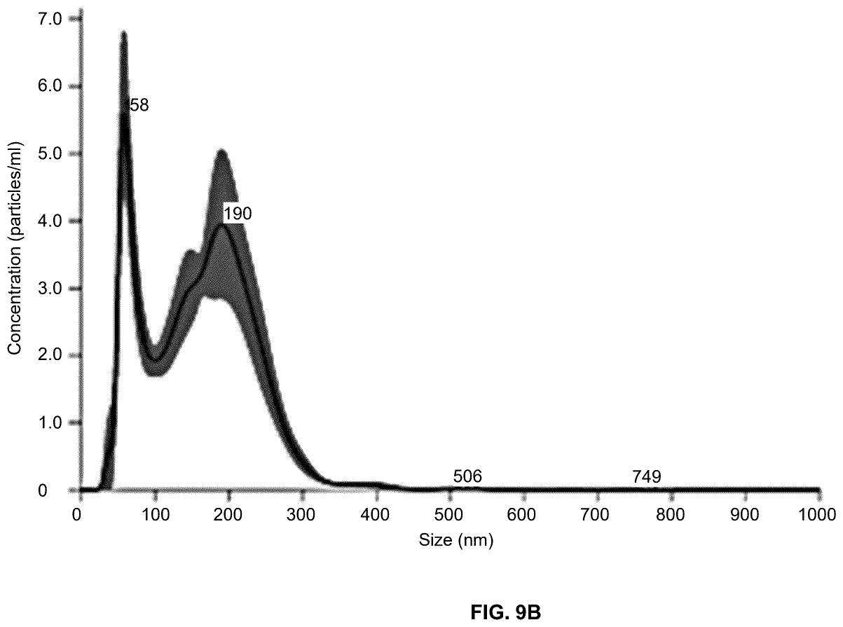

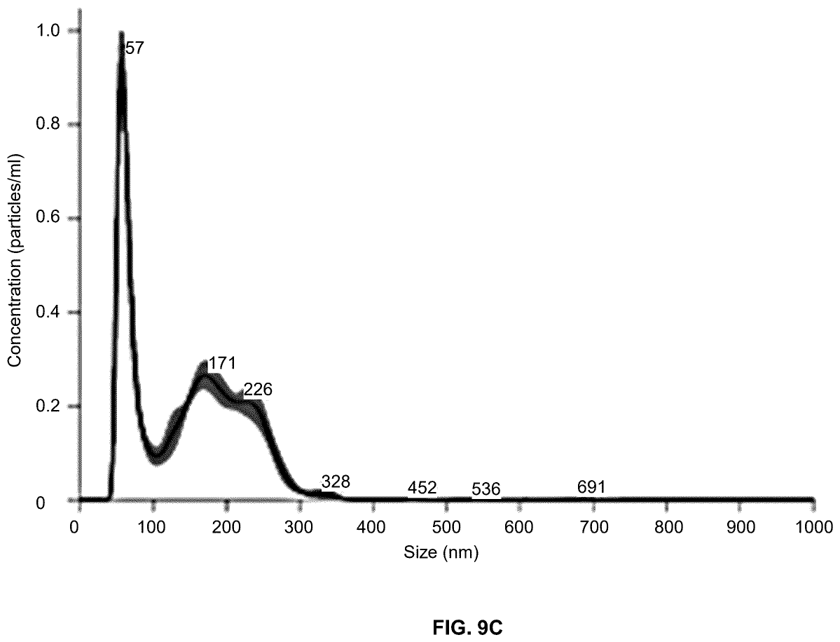

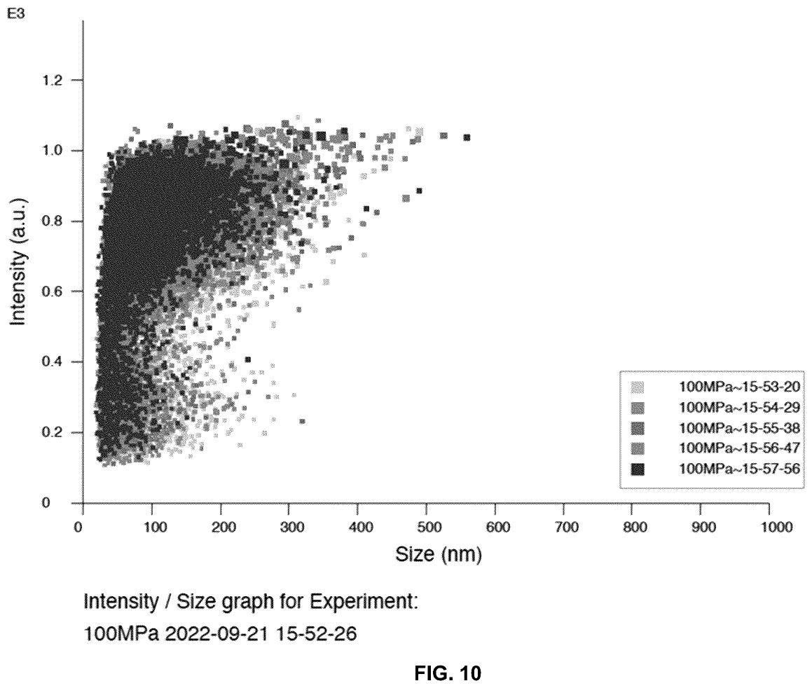

Particle size and concentration were quantified using a Malvern Panalytical NS300 Nanoparticle Tracking Analyzer (Malvern, UK). Samples were diluted in phosphate buffered saline and vortexed to mix immediately prior to the experiment. Five, 60-second measurements were collected at a flow rate of 70 μl/min for each sample. Examples of particle sizes are shown for homogenization conditions under 100, 200, and 300 MPa, as shown in FIGS. 9A-9C. Particles made under 100 MPa were stored under 4° C. and retested at 30 days, which confirmed no particles larger than 400 nm in size, as shown in FIG. 10.

### Further Size Stability Testing

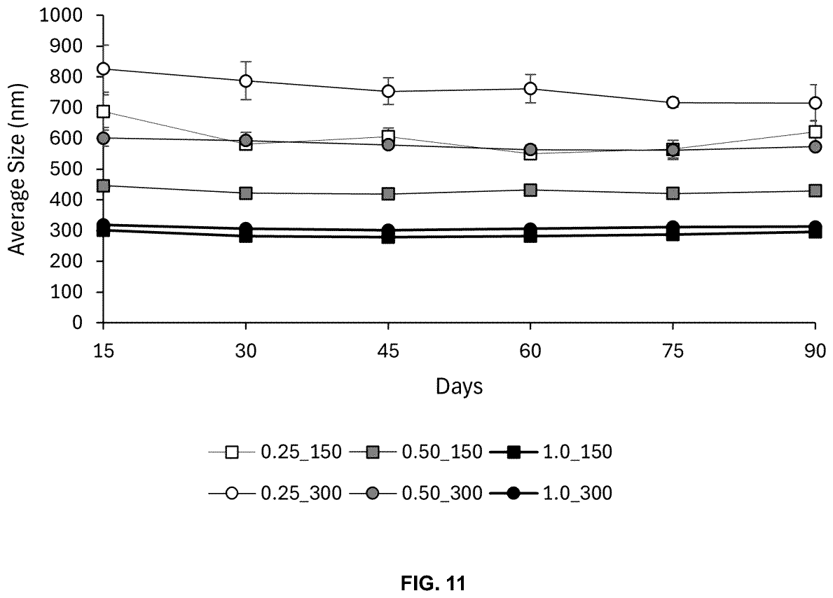

We measured the size stability of our emulsions for several months by monitoring particle size. Emulsions were generated from 20% fractionated milkfat in water with 0.25, 0.5, or 1.0% polysorbate 80 emulsifier, and 150 or 300 MPa high-pressure homogenization (both pressures establish a suitable particle size) for three passes (i.e., sent through the homogenizer three times). The emulsions were stable (particle size ˜300 nm) for over three months (FIG. 11, Tables 8A and 8B).

TABLE 8A

| Samples | | | | |
|---|---|---|---|---|
| | Homog- | | | |
| Emulsifier | enization | | | |
| Conc. | pressure | Days | | |
| (% w/w) | (mPa) | 15 | 30 | 45 |
| 0.25 | 150 | 688.13 ± 52.69 | 580.67 ± 13.80 | 605.03 ± 29.13 |
| 0.50 | 150 | 445.93 ± 4.55 | 421.70 ± 7.39 | 419.20 ± 4.09 |
| 1.0 | 150 | 301.50 ± 16.74 | 282.23 ± 15.56 | 279.10 ± 11.47 |
| 0.25 | 300 | 826.17 ± 76.17 | 786.70 ± 61.91 | 753.23 ± 44.03 |
| 0.50 | 300 | 600.50 ± 26.15 | 593.17 ± 26.81 | 578.93 ± 10.64 |
| 1.0 | 300 | 319.30 ± 6.25 | 306.13 ± 7.09 | 300.63 ± 5.55 |

Values shown as mean size (nm) ± StDev (n = 3)

TABLE 8B

| Samples | | | | |
|---|---|---|---|---|
| | Homog- | | | |
| Emulsifier | enization | | | |
| Conc. | pressure | Days | | |
| (% w/w) | (mPa) | 60 | 75 | 90 |
| 0.25 | 150 | 549.47 ± 14.10 | 564.60 ± 29.40 | 621.27 ± 36.99 |
| 0.50 | 150 | 431.87 ± 2.32 | 420.37 ± 5.78 | 429.40 ± 6.24 |
| 1.0 | 150 | 282.50 ± 19.50 | 287.97 ± 17.98 | 296.30 ± 14.60 |
| 0.25 | 300 | 761.73 ± 46.01 | 716.13 ± 14.10 | 714.47 ± 59.12 |
| 0.50 | 300 | 563.27 ± 14.93 | 561.93 ± 31.86 | 572.43 ± 10.31 |
| 1.0 | 300 | 305.50 ± 5.38 | 311.20 ± 3.82 | 312.47 ± 8.86 |

Values shown as mean size (nm) ± StDev (n = 3)

These emulsions are also stable to autoclaving, which is excellent for sterility. We tested emulsifier concentrations of 0.25 and 0.50% (w/w). The particles were much larger (>400 nm) with these concentrations.

### In Vitro Inflammatory Response

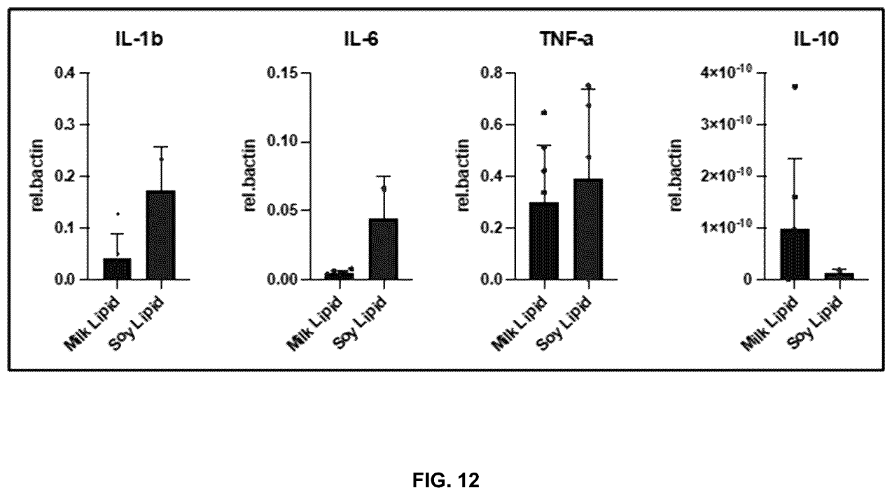

The IVFEs prepared from LMF-25 and processed at 300 MPa first-stage pressure were compared with soy-lipid Intralipid® 20% for inflammatory effects on bone marrow derived macrophages in vitro. The milk lipid IVFEs had less of an inflammatory response (IL-1b, IL-6, and TNF-α) than the soy lipid IVFEs and induced an anti-inflammatory response with IL-10 (FIG. 12).

### Intravenous Administration and Metabolic Response In Vivo

Following intravenous catheterization under sterile technique, animals were administered parenteral nutrition (PN) and experimental lipid emulsions at 10-15% (v/v). The IVFEs prepared from LMF-25 and processed at 300 MPa first-stage pressure were selected as they achieved the minimum emulsion droplet size and were abundant in medium-chain triglycerides. Administration rates were based on animal body weight, tolerability, and calculated to meet caloric, nitrogen, and fat requirements for the animals.

All animal experiments were approved by IACUC at the University of Wisconsin-Madison. Male wild-type C57Bl/6 mice housed under standard 12:12 light/dark conditions. Mice were 8-10 weeks of age and maintained in microisolator cages for microbiome containment and fidelity. Animals were weighed, provided buprenorphine ER analgesics (0.6/mg/kg), anesthetized by isoflurane, and underwent placement of silicon rubber catheter (0.012-inch I.D./0.025-inch O.D.; Helix Medical, Inc., Carpinteria, CA) in the vena cava through the right external jugular vein. Intravenous catheters were tunneled subcutaneously and dorsally at the midpoint between the scapulae. Animals were harnessed and tethered in individually housed metabolic cages with continuous gas exchange monitoring.

Intravenous formulation: Cannulated mice were connected to infusion pumps and intravenous solution was provided at 18, 25 and 32% volume/body weight per day over the first 3 days, with 32% volume/body weight maintained for the final 3 days. At 48 hours, 2% weight/volume fat was increased to 3% weight/volume for all animals. The PN solution contained 1170 kcal/L consisting of 5.0% amino acids, 25% dextrose, 2-3% fat by volume. A standard control IVFE was provided with 20% v/v Intralipid-20 (emulsion, Baxter Healthcare Corporation, Deerfield, IL) and compared with IVFEs prepared from LMF-25 and processed at 300 MPa first-stage pressure. These IVFEs were selected as they achieved the minimum emulsion droplet size and were abundant in medium-chain triglycerides. Intravenous solutions were replaced daily to prevent lipid micelle precipitation. After 6 days, animals were humanely euthanized for collection of serum, tissues, and microbiome samples were collected for analysis.

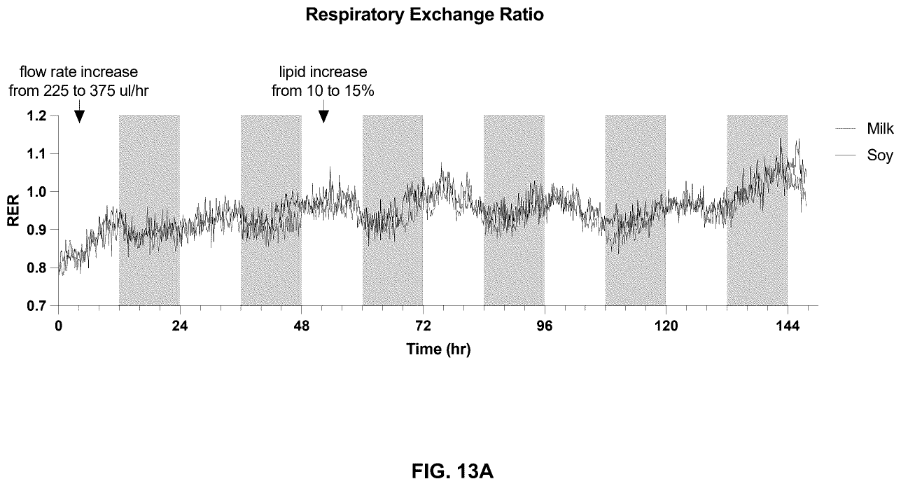

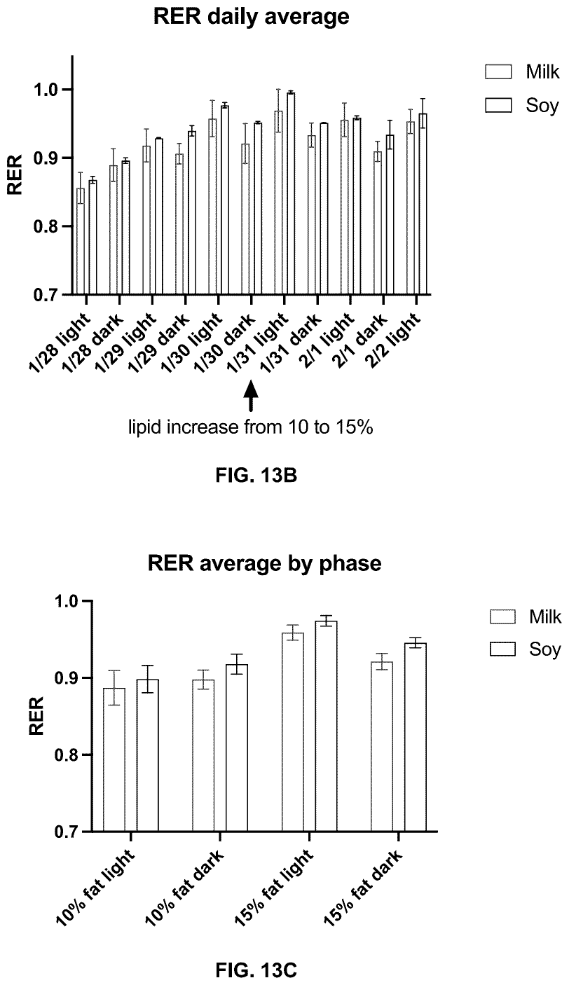

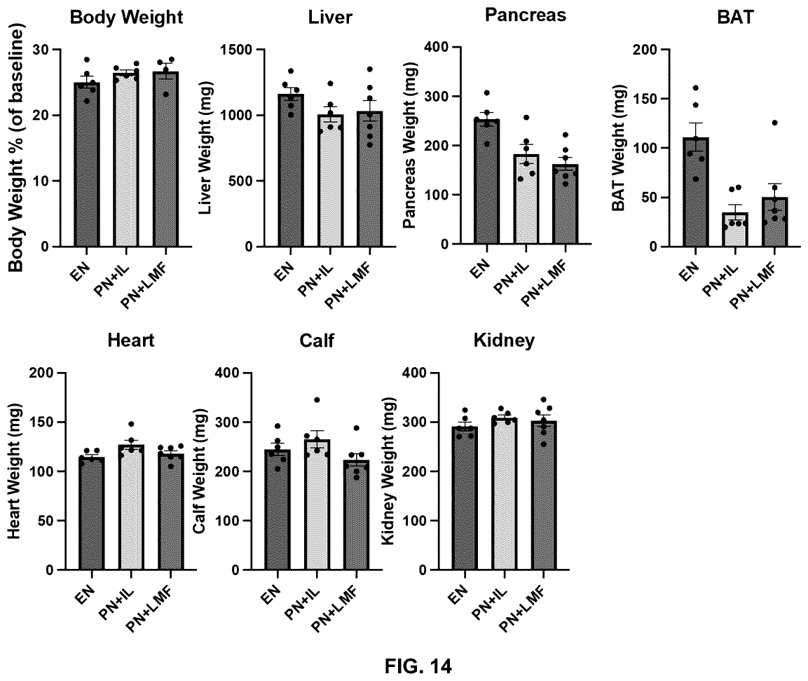

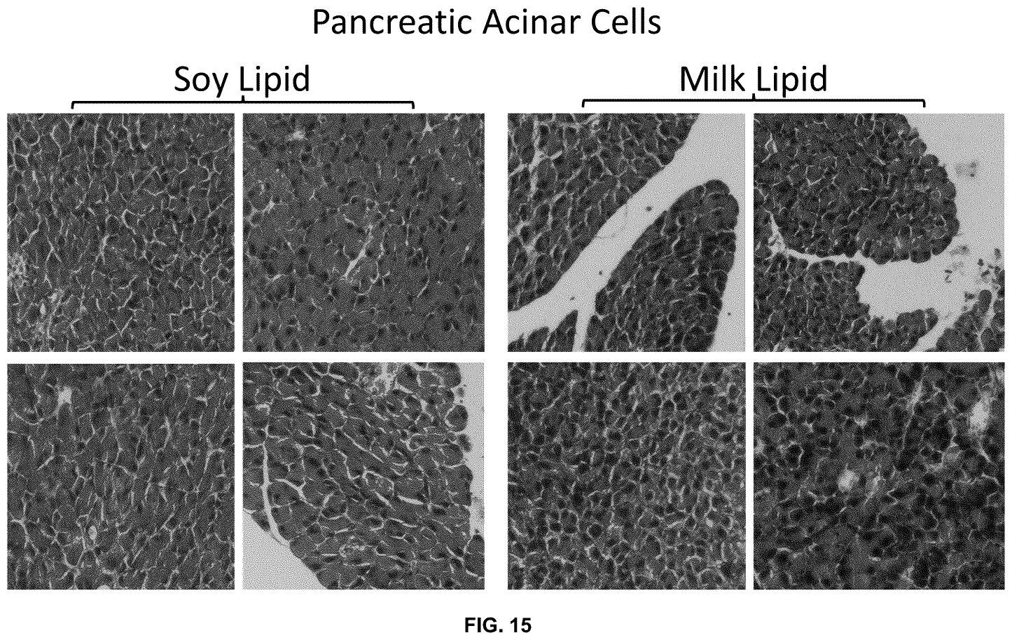

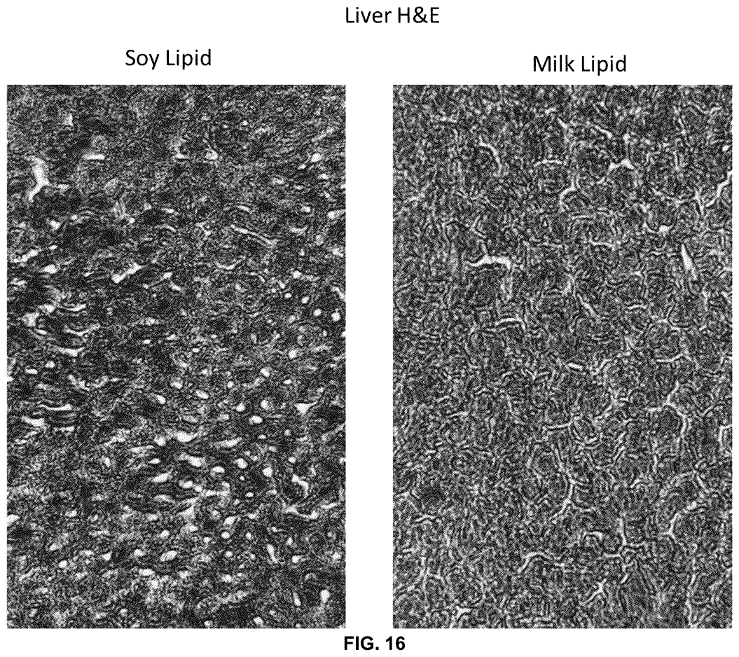

FIGS. 13A-13C show respiratory exchange ratio (RER) of the mice, where LMF decreased RER levels compared with Intralipid, supporting enhanced lipid utilization in metabolism. FIG. 14 shows body, organ and tissue weights. FIG. 15 shows hematoxylin and eosin staining of pancreatic acinar tissue, demonstrating smaller acinar cell size in response to LMF emulsions compared with Intralipid. FIG. 16 shows hematoxylin and eosin staining of liver tissue.

### CONCLUSIONS

The approach outlined herein involves fractionation and high-pressure homogenization methodologies to achieve stable milkfat microemulsions for intravenous applications. When provided in preclinical models, milk fat microemulsions lead to comparable or superior survival and potential improvements in digestive organ function homeostasis without evidence of systemic inflammation. Hence, the milkfat microemulsions provided herein provide a satisfactory source of intravenous lipids for patients requiring parenteral nutrition and will contribute to new pharmaceutical applications for dairy lipids markets.

### REFERENCES

ADDIN EN. REFLIST Ahmad, S., M. Nadeem, M. Ayaz, and M. H. Jaspal. 2015. Effect of Low-Melting Fractions of Milk Fat on Lipolysis of Cheddar Cheese. Journal of food processing and preservation 2015 v.39 no. 6 (no. 6): pp. 2516-2522.
Anez-Bustillos, L., D. T. Dao, M. A. Baker, G. L. Fell, M. Puder, and K. M. Gura. 2016. Intravenous Fat Emulsion Formulations for the Adult and Pediatric Patient: Understanding the Differences. Nutr Clin Pract 31 (5): 596-609.
Atkins, N. E., C. Cianchi, S. M. Rutter, S. J. Williams, C. Gauld, G. L. Charlton, and L. A. Sinclair. 2020. Performance, milk fatty acid composition and behaviour of high-yielding Holstein dairy cows given a limited grazing period. Grass and Forage Science 75 (2): 181-191.
Bailey, K. W., C. M. Jones, and A. J. Heinrichs. 2005. Economic Returns to Holstein and Jersey Herds Under Multiple Component Pricing. Journal of Dairy Science 88 (6): 2269-2280.
Barca, J., M. Carriquiry, O. Laura, M. Fajardo, P. Chilibroste, and A. Meikle. 2017. Milk fatty acid profile from cows fed with mixed rations and different access time to pastureland during early lactation. Journal of Animal Physiology and Animal Nutrition 102.
Chandan, R. C. 2015. Dairy Processing and Quality Assurance: An Overview. Pages 1-40 in Dairy Processing and Quality Assurance.
Coffey, E. L., B. Horan, R. D. Evans, and D. P. Berry. 2016. Milk production and fertility performance of Holstein, Friesian, and Jersey purebred cows and their respective crosses in seasonal-calving commercial farms. Journal of Dairy Science 99 (7): 5681-5689.
Diels, A. M. and C. W. Michiels. 2006. High-pressure homogenization as a non-thermal technique for the inactivation of microorganisms. Crit Rev Microbiol 32 (4): 201-216.
Floury, J., J. Legrand, and A. Desrumaux. 2004. Analysis of a new type of high pressure homogeniser. Part B. study of droplet break-up and recoalescence phenomena. Chemical Engineering Science 59 (6): 1285-1294.
Galvão, K. C. S., A. A. Vicente, and P. J. A. Sobral. 2018. Development, Characterization, and Stability of O/W Pepper Nanoemulsions Produced by High-Pressure Homogenization. Food and Bioprocess Technology 11 (2): 355-367.
Hardy, G. and M. Puzovic. 2009. Formulation, stability, and administration of parenteral nutrition with new lipid emulsions. Nutr Clin Pract 24 (5): 616-625.
Harte, F. 2016. Food Processing by High-Pressure Homogenization. Pages 123-141 in High Pressure Processing of Food: Principles, Technology and Applications. V. M. Balasubramaniam, G. V. Barbosa-Cánovas, and H. L. M. Lelieveld, ed. Springer New York, New York, NY.
Harte, F., M. Amonte, L. Luedecke, B. G. Swanson, and G. V. Barbosa-Cánovas. 2002. Yield Stress and Microstructure of Set Yogurt Made from High Hydrostatic Pressure-Treated Full Fat Milk. Journal of Food Science 67 (6): 2245-2250.
Harte, F. and R. Venegas. 2010. A model for viscosity reduction in polysaccharides subjected to high-pressure homogenization. Journal of Texture Studies 41:49-61.
Hayes, M. G. and A. L. Kelly. 2003. High pressure homogenisation of raw whole bovine milk (a) effects on fat globule size and other properties. Journal of Dairy Research 70 (3): 297-305.
Illingworth, D., G. R. Patil, and A. Y. Tamime. 2009. Anhydrous Milk Fat Manufacture and Fractionation. Pages 108-166 in Dairy Fats and Related Products.
Jadhav H B, Annapure U S. Triglycerides of medium-chain fatty acids: a concise review. J Food Sci Technol. 2023 August; 60 (8): 2143-2152. doi: 10.1007/s13197-022-05499-w. Epub 2022 Jun. 22.
Kailasapathy, K. 2015. Chemical Composition, Physical, and Functional Properties of Milk and Milk Ingredients. Pages 77-105 in Dairy Processing and Quality Assurance.
Rico, D. E., E. R. Marshall, J. Choi, K. E. Kaylegian, C. D. Dechow, and K. J. Harvatine. 2014. Within-milking variation in milk composition and fatty acid profile of Holstein dairy cows. Journal of Dairy Science 97 (7): 4259-4268.
Shi, S., H. Chen, Y. Cui, and X. Tang. 2009. Formulation, stability and degradation kinetics of intravenous cinnarizine lipid emulsion. Int J Pharm 373 (1-2): 147-155.

1. A method of generating lipid particles, the method comprising:
generating a combined composition, comprising combining a milk fat composition comprising a solids portion comprising target milk fat with a surfactant; and
generating a lipid-particle composition comprising lipid particles, comprising emulsifying the combined composition.

2. The method of claim 1, wherein the solids portion comprises the target milk fat in amount of at least 50% w/w.

3. The method of claim 1, wherein the lipid composition comprises the solids portion in an amount of at least 50% w/w.

4. The method of claim 1, wherein the lipid particles comprise triglycerides in an amount greater than 50% w/w of combined total of triglyceride, diglyceride, 1,2-diacylglyceryl-3-O-4′-(N,N,N-trimethyl)-homoserine, phosphatidylcholine, ether-linked phosphatidylcholine, fatty acyl ester of hydroxy fatty acid, free fatty acid, lysophosphatidylcholine, phosphatidylethanolamine, ether-linked phosphatidylethanolamine, lysophosphatidylethanolamine, non-hydroxy-fatty acid sphingosine ceramide, and sphingomyelin detected in positive ion mode of ultra-high-performance liquid chromatography/mass spectrometry/mass spectrometry.

5. The method of claim 1, wherein the lipid particles comprise a relative amount of any 3 or more of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, and TG 12:0_14:0_16:0.

6. The method of claim 1, wherein the lipid particles comprise a relative amount of any 10 or more of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, TG 12:0_14:0_16:0, TG 16:0_18:0_18:1, TG 16:0_18:1_18:1, TG 16:0_16:0_18:1, TG 14:0_16:0_18:1, TG 12:0_16:0_18:1, TG 12:0_14:0_18:1, TG 14:0_18:1_18:1, TG 18:0_18:0_18:1, TG 16:0_17:0_18:1, TG 18:1_18:1_18:1, TG 12:0_12:1_18:1, TG 15:0_18:1_18:1, TG 15:0_16:0_18:1, TG 17:0_18:1_18:1, TG 17:0_18:0_18:1, TG 16:0_18:1_18:3, TG 16:0_18:0_18:0, TG 14:0_18:1_18:2, TG 14:0_16:0_18:0, TG 12:0_15:0_18:1, TG 15:0_16:0_18:0, and TG 16:0_17:0_18:0.

7. The method of claim 1, wherein the combined composition comprises the solids portion of the milk fat composition in an amount of at least 2.5% w/w.

8. The method of claim 1, wherein the generating the combined composition further comprises combining the milk fat composition with water.

9. The method of claim 1, wherein the combined composition comprises water in an amount of at least 50% w/w.

10. The method of claim 1, wherein the combined composition comprises the surfactant in an amount of at least 0.1% w/w and/or up to 10% w/w.

11. The method of claim 1, wherein greater than 90% by number of the lipid particles in the lipid-particle composition have a diameter of less than 500 nm.

12. The method of claim 1, wherein the emulsifying comprises high-pressure homogenization.

13. The method of claim 1, wherein the emulsifying generates an emulsified composition and the generating the lipid-particle composition further comprises size-filtering the emulsified composition with a filter comprising a pore size from 0.5 μm to 5.0 μm.

14. The method of claim 1, further comprising autoclaving the lipid particles.

15. The method of claim 1, further comprising generating the milk fat composition from a prior milk fat composition comprising the target milk fat and additional milk fat by removing the additional fat from the target milk fat.

16. The method of claim 15, wherein the generating the milk fat composition comprises melt fractionating the prior milk fat composition.

17. The method of claim 15, wherein the prior milk fat composition comprises at least one of anhydrous milk fat, butter oil, and ghee.

18. The method of claim 1, wherein the target milk fat comprises ruminant milk fat.

19. The method of claim 1, wherein:
the solids portion comprises the target milk fat in amount of at least 50% w/w;
the lipid composition comprises the solids portion in an amount of at least 50% w/w;
the lipid particles comprise triglycerides in an amount greater than 50% w/w of combined total of triglyceride, diglyceride, 1,2-diacylglyceryl-3-O-4′-(N,N,N-trimethyl)-homoserine, phosphatidylcholine, ether-linked phosphatidylcholine, fatty acyl ester of hydroxy fatty acid, free fatty acid, lysophosphatidylcholine, phosphatidylethanolamine, ether-linked phosphatidylethanolamine, lysophosphatidylethanolamine, non-hydroxy-fatty acid sphingosine ceramide, and sphingomyelin detected in positive ion mode of ultra-high-performance liquid chromatography/mass spectrometry/mass spectrometry;
the lipid particles comprise a relative amount of any 3 or more of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, and TG 12:0_14:0_16:0;
the lipid particles comprise a relative amount of any 10 or more of the following lipids within 5× of each other as detected in positive ion mode of ultra-high-performance liquid chromatography/mass spectrometry/mass spectrometry: TG 14:0_16:0_16:0, TG 14:0_15:0_16:0, TG 12:0_16:0_16:0, TG 12:0_15:0_16:0, TG 12:0_14:0_16:0, TG 16:0_18:0_18:1, TG 16:0_18:1_18:1, TG 16:0_16:0_18:1, TG 14:0_16:0_18:1, TG 12:0_16:0_18:1, TG 12:0_14:0_18:1, TG 14:0_18:1_18:1, TG 18:0_18:0_18:1, TG 1_16:0_17:0_18:1, TG 18:1_18:1_18:1, TG 12:0_12:1_18:1, TG 15:0_18:1_18:1, TG 15:0_16:0_18:1, TG 17:0_18:1_18:1, TG 17:0_18:0_18:1, TG 16:0_18:1_18:3, TG 16:0_18:0_18:0, TG 14:0_18:1_18:2, TG 14:0_16:0_18:0, TG 12:0_15:0_18:1, TG 15:0_16:0_18:0, and TG 16:0_17:0_18:0;
the combined composition comprises the solids portion of the milk fat composition in an amount of at least 7.5% w/w;
the generating the combined composition further comprises combining the milk fat composition with water;
the combined composition comprises water in an amount of at least 60% w/w;
the combined composition comprises the surfactant in an amount of at least 0.1% w/w and up to 10% w/w;
greater than 90% by number of the lipid particles in the lipid-particle composition have a diameter of less than 500 nm;
the emulsifying comprises high-pressure homogenization;
the emulsifying generates an emulsified composition and the generating the lipid-particle composition further comprises size-filtering the emulsified composition with a filter comprising a pore size from 0.5 μm to 5.0 μm;
the method further comprises autoclaving the lipid particles;
the method further comprises generating the milk fat composition from a prior milk fat composition comprising the target milk fat and additional milk fat by removing the additional fat from the target milk fat;
the generating the milk fat composition comprises melt fractionating the prior milk fat composition;
the prior milk fat composition comprises at least one of anhydrous milk fat, butter oil, and ghee; and
the target milk fat comprises ruminant milk fat.

20. Lipid particles made from the method of claim 1.

21. A method of administering parenteral nutrition, the method comprising parenterally administering the lipid particles of claim 20 to a subject, optionally, wherein the subject has a condition comprising at least one of pancreatitis and hepatitis, optionally, wherein the administering results in a reduced pro-inflammatory response relative to administering an equivalent amount of lipid particles generated from vegetable fat.
